(19) United States
(12) Patent Application Publication
(10) Pub. No.: US 2022/0389389 A1
(43) Pub. Date: Dec. 8, 2022

(54) METHODS AND A KIT TO REPROGRAM SOMATIC CELLS

(71) Applicant: Novoxyne LLC, Fair Lawn, NJ (US)

(72) Inventor: Atsuo OCHI, Fair Lawn, NJ (US)

(21) Appl. No.: 17/775,801

(22) PCT Filed: Nov. 11, 2020
(86) PCT No.: PCT/US2020/060071

§ 371 Date: May 10, 2022

Related U.S. Application Data
(60) Provisional application No. 62/933,926, filed on Nov. 11, 2019.

(51) Int. Cl. C12N 5/074; C12M 1/12; A61K 35/12

(52) U.S. Cl. CPC C12N 5/0696; C12M 25/04; A61K 35/12; C12N 2506/11; C12N 2501/998; C12N 2501/999; C12N 2533/30; C12N 2533/70

(57) ABSTRACT

The present invention relates to methods for reprogramming somatic cells into pluripotent stem cell-like cells. Such cells may express pluripotency inducing genes including Oct4, Nanog and Sox2 without introducing exogeneous genes, proteins, or chemicals. The discovery that the inhibition of mechanosensitive and stretch-activated ion channels in somatic cells specifically activates pluripotency inducing factor genes inspired the cell reprogramming culture methods in which somatic cells were incubated with the inhibitor, GsMTX4, against mechanosensitive and stretch-activated ion channels, cultured on the soft hydrogel surface, or treated with cholesterol depletion substance, methyl-beta-cyclodextrin (MβCD). Described methods produce pluripotent stem cell-like cells and subsequently re-differentiated cells, which include adipocytes, osteocytes, neuronal cells. Methods may be combined to increase the efficiency of the somatic cell reprogramming A somatic cell reprogramming kit was also created with tissue culture dishes casted with hydrogel (dehydrated) and MβCD.

### 1. CROSS-REFERENCE TO RELATED APPLICATION

This application claims priority to U.S. Provisional Patent Application No. 62/933,926 filed Nov. 11, 2019.

### 2. SEQUENCE LISTING

The application contains a Sequence Listing which has been submitted electronically in ASCII format and is hereby incorporated by reference in its entirety. Said ASCII copy, created on Nov. 11, 2020, is named 001WO_SL_ST25.txt and is 26,627 bytes in size.

### 3. FIELD OF THE INVENTION

The invention generally relates to methods of reprogramming somatic cells to become pluripotent stem cell-like cells possessing differentiation potentials. The described methods, reagents, compositions, stem cell-like cells and the differentiated cells such as neuronal cells, fat cells, muscle cells and bone cells are used for treating various diseases requiring regenerative therapies.

### 4. BACKGROUND OF THE INVENTION

Affording to current understanding of molecular and cellular mechanisms of degenerative diseases, pluripotent stem cells and the differentiated cells are in great demand to meet the progress of regenerative medicine.

Under the homeostatic mechanism of multi-cellular organism, the terminally differentiated and damaged cells are removed by programmed cell death, while those removed are promptly replaced with newly differentiated cells possessing the same function. When the cell death surpasses the generation of new cells in the pathogenic circumstance, or by the tissue injuries where the large proportion of the tissues is lost, regenerative therapy becomes vital to recover the lost tissues to restore the organ functions. The discovery of the presence of stem cells in blastocyst, which possess pluripotent differentiation potential, prompted the challenge to establish the regenerative stem cell therapy, which can generate the differentiated cells to rebuild the lost parts of the organ or whole organ. Listed below are cell sources currently recognized as possessing differentiation potentials through manipulation ex vivo, to generate various types of cells useful to apply for regenerative therapy.

Embryonic Stem Cells (ES Cells)

ES cells are pluripotent cells derived from human or mouse early embryos, blastocysts [Evans and Kaufman, Nature, 292:154 (1981); Martin, Proc. Natl. Acad. Sci. USA, 78:7634 (1981)], which have a unique feature that they can be in vitro cultured over a long period of time while maintaining ability to differentiate into all kinds of cells existing in living bodies. Human embryonic stem cells are expected to be useful for cell transplantation therapies for various diseases such as Parkinson's disease, juvenile diabetes, and leukemia, taking advantage of the above described properties. However, transplantation of ES cells has a problem of causing rejection in the same manner as organ transplantation. Moreover, from an ethical viewpoint, there are many dissenting opinions against the use of ES cells which are established by destroying human embryos.

Hematopoietic Stem Cells

A hematopoietic stem cell is a cell isolated from the blood or bone marrow that can renew itself, can differentiate to a variety of specialized cells, can mobilize out of the bone marrow into circulating blood, and can undergo programmed cell death, by which cells that are detrimental or unneeded self-destruct. About 1 in every 100,000 cells in the marrow is a long-term, blood-forming stem cell; other cells present in the bone marrow include stromal cells, stromal stem cells, blood progenitor cells, and mature and maturing white and red blood cells.

Mesenchymal Stem Cells (MSCs)

MSCs are an example of tissue or ‘adult’ stem cells including bone marrow stromal cells and umbilical cord cells and fat derived stromal/stem cells. They are ‘multipotent’, meaning they can produce more than one type of specialized cell of the body, but not all types. MSCs make the different specialized cells found in the skeletal tissues. For example, they can differentiate—or specialize—into cartilage cells (chondrocytes), bone cells (osteoblasts) and fat cells (adipocytes). These specialized cells each have their own characteristic shapes, structures and functions, and each belongs in a particular tissue. Although the differentiation potential of the MSCs suggests that they may be useful for cell therapy in various diseases such as myocardial repair, and bone and neuronal repairs, the expansion of the purified mesenchymal stem cells is not consistent between the individuals, age and the origin of the tissue. Moreover, the proliferation of the cells declines after several passages and the preparation of a large number of the cells is problematic.

Induced Pluripotent Stem Cells

If dedifferentiation of patients' own differentiated somatic cells could be induced to establish cells having pluripotency and growth ability similar to those of ES cells, it is anticipated that such induced pluripotent stem cells (iPSCs) could be used as ideal pluripotent cells, free from rejection or ethical difficulties [U.S. Pat. No. 10,017,744]. The iPSCs, originally reported by the Yamanaka's group [Takahashi and Yamanaka, Cell 126: 663 (2006); U.S. Pat. No. 8,048,999], are mouse adult cells that have been genetically reprogrammed to an embryonic stem cell-like state by being forced to express genes and factors important for maintaining the defining properties of embryonic stem cells. Human iPSCs were first reported in late 2007 [Takahashi et al., Cell 131: 861 (2007); U.S. Patent Publication No. 2013/0065311]. Mouse iPSCs demonstrated important characteristics of pluripotent stem cells, including expressing stem cell markers, forming tumors containing cells from all three germ layers, and being able to contribute to many different tissues when injected into mouse embryos at a very early stage in development. Human iPSCs also express stem cell markers and are capable of generating cells characteristic of all three germ layers. Although iPSCs meet the defining criteria for pluripotent stem cells, it is not known if iPSCs and embryonic stem cells differ in clinically significant ways.

Moloney Murine Leukemia virus and Lentivirus were originally used to introduce reprogramming factors into adult cells [U.S. Pat. No. 8,440,461]. Because this process results in the integration of the virus, it must be regulated and tested before the technique can lead to useful treatment for humans to avoid possible development of cancers. In animal studies, the virus used to introduce the stem cell factors sometimes causes cancers. Adenovirus and Sendai virus were non-integrating virus and also used to introduce pluripotency inducing factors (PIFs) into adult cells, although the reprogramming efficiency is low [Zhou and Freed, Stem Cells. 27: 2667 (2009); Chen et al., Cell Reprogram. 15: 503 (2013)]. Challenges by researchers to innovate viral infection and genome integration-based reprogramming methods to non-viral delivery strategies resulted in the alternate technologies to induce the transient pluripotency inducing gene expression in somatic cells. Those are reprogramming factor delivery system using Episome, RNA and recombinant proteins, although each has disadvantages such as low efficiency or difficulty in preparation (for protein-based delivery system) [Woltjen et al., Nature 458: 766 (2009); U.S. Publication No. 2018/0072999; Kogut I. et al. Nature Communications 9: 745 (2018); U.S. Publication No. 2013/0302295; International Patent Publication No. WO2009/077134; Zhou H. et al., Cell Stem Cell 4: 381 (2009); U.S. Pat. No. 9,068,170]. These techniques allow one to “de-differentiate” cells whose developmental fates had been previously assumed to be determined. The iPSC technology, by introducing reprogramming factors, offers the opportunity to generate patient-specific stem cells for modeling human diseases, drug development and screening, and individualized regenerative cell therapy.

Chemically Assisted and Reprogrammed Somatic Cells (ciPSCs)

The use of viral vectors and oncogenes for PI gene expression has generated valid concerns over the safe use of these cells in clinics. Thus, the field has shifted towards new chemical/small molecule (<900 Daltons)-based reprogramming strategies. The first reported is the chemical assisted generation of ciPSC, in which, by using the histone deacetylase inhibitor Valproic acid (VPA), eliminated the need for oncogenes c-Myc and Klf4 (two of the four Yamanaka factors), and also found that the reprogramming efficiency was increased 100-fold over that of the four transcription factor method [Huangfu et al., Nat. Biotechnol., 26: 1269 (2008); U.S. Pat. No. 9,982,237]. On the other, the studies used the histone methyltransferase (HMT) inhibitor BIX-01294, to activate calcium channels in the plasma membrane, and improved the reprogramming efficiency using the four Yamanaka factors from Ding's laboratory [Armond et al., Sci. Rep., 4: 3692 (2014); Shi et al., Cell Stem Cell, 3: 568 2008); Shi et al., Cell Stem Cell, 2: 525 (2008); Lin et al., Nat. Methods., 6: 805 (2009)]. The inhibitors of transforming growth factor-β (TGFβ) receptor and MAPK/ERK kinase (MEK) on primary human fibroblasts (CRL2097 or BJ) that were transduced with retrovirus carrying genes encoding the four Yamanaka factors [Australian Patent Application No. 2015201026]. They showed that a combination of seven small molecules improved the efficiency of iPSC generation from human fibroblasts by greater than 200 fold within a week of treatment. Subsequently, several groups identified specific chemical combinations, which were sufficient to permit reprogramming from mouse embryonic and adult fibroblasts in the presence of a single transcription factor, Oct4, within three weeks, without the need for Sox2, Klf4 and c-Myc [Li et al., Cell Res., 21: 196 (2011); Yuan et al., Stem Cells, 29: 549 (2011); Zhu et al., Cell Stem Cell, 7: 651 (2010)]. The iPSCs developed using this protocol are similar to mouse ES (mES) cells in terms of expression of pluripotency genes, epigenetic state, and global gene expression profile.

Hou et al. were the first to report all chemical generation of mouse iPSCs from mouse embryonic fibroblasts (MEFs) at a efficiency up to 0.2% using a combination of seven small-molecule compounds VC6TFZ: VPA, CHIR99021 (CHIR), 616452, Tranylcypromine, Forskolin (FSK), 2-methyl-5-hydroxytryptamine (2-Me-5HT), and D4476 [Hou et al., Science, 341: 651 (2013); International Patent Publication No. WO2015/003643]. This method also had a higher efficiency of induction compared to Yamanaka's iPSC protocol (0.01%-0.1%). The chemically induced pluripotent stem cells (ciPSCs) exhibited similar global gene expression profiles as mES cells. This study provided the proof of principle that by using small molecules, ectopic expression of master regulator genes is not necessary for cell fate reprogramming, thus showing the way for all-chemical reprogramming strategy with potential use in generating functionally desirable cell types for cell therapy. Most small molecules that have been used so far to generate ciPSCs can be categorized as epigenetic modifiers, modifiers of cell signaling and apoptosis, wingless and integration site growth factor (MINT) signal modulators, moderators of cell senescence, or modulators of metabolism.

The Stem Cell Like Phenotype Cells Induced by the Specific Fashion of Cell Cultivation

Nuclear reprogramming events within tissue microenvironments are critical for a number of developmental processes and tissue maintenance [Halley-Stott et al., Development, 140: 2468 (2013); Reik et al., Science, 293:1089 (2001); Lamouille et al., Nat. Rev. Mol. Cell. Biol., 15: 178 (2014)]. In landmark experiments, biochemical factors were shown to induce nuclear reprogramming of somatic cells into iPSC in vitro [Takahashi and Yamanaka, Cell, 126: 663 (2006); De Matteis et al., Stem Cells, 27: 2761 (2009); Downing et al., Nat. Mater., 12: 1154 (2013)]. However, in vivo, cells transdifferentiate into different lineages in the absence of exogenous factors, indicating that the local mechanochemical factors could be important elements and are sufficient for inducing such transitions [Guo et al., Proc. Natl. Acad. Sci. USA, 111: 5252 (2014)]. Consistent with this, recent results have shown that culturing cells on topographic patterns combined with reprogramming factors, or on different substrate rigidity, resulted in increased efficiency of nuclear reprogramming [Su, et al., Biomaterials, 34: 3215 (2013); Kilian et al., Proc. Natl. Acad. Sci. USA, 107: 4872 (2010); Engler et al., Cell, 126: 677 (2006); Mitra et al., Proc. Natl. Acad. Sci. USA, 114: 3882 (2017)]. However, the roles played by different mechanical cues in the absence of biochemical factors in nuclear reprogramming has not been well established. Mechanical constraints (e.g., substrate rigidity and cell morphology) are essential in controlling many cellular processes, including cellular proliferation, apoptosis, and differentiation [Chen et al., Science, 276:1425 (1997); McGrail et al., FASEB J., 29:1280 (2015); Kshitiz et al., Integr Biol., 4:1008 (2012); Kumar et al., PLoS One 7:e33089 (2012)]. Biophysical forces have been shown to be important in regulating epithelial to mesenchymal transformation [Desprat et al., Dev Cell, 15: 470 (2008)]. Matrix rigidity, cell shape, and surface topography all have been shown to direct stem cell differentiation in vitro [McGrail et al., FASEB J., 29:1280 (2015); Kshitiz et al., Integr Biol., 4:1008 (2012); U.S. Patent Publication No. 2008/0187995]. In addition, in vivo experiments involving applying forces on a developing Drosophila embryo have demonstrated that altering the mechanics of the tissue can alter the differentiation programs [Kumar et al., PLoS One, 7:e33089A (2012); Desprat et al., Dev Cell, 15: 470 (2008)]. Collectively, these results highlight the importance of biophysical cues in directing stem cell differentiation.

As used herein the following terms shall have the meanings set forth. Otherwise all terms shall have the meaning they would normally be accorded by a person skilled in the relevant art.

The term “Blastocyst” refers to a thin-walled hollow structure in early embryonic development that contains a cluster of cells called the inner cell mass from which the embryo arises.

The term “Pluripotency inducing gene” refers to a gene whose expression, contributes to reprogramming somatic cells to a pluripotent state.

The term “Pluripotency inducing factor” refers to an expression product of a pluripotency inducing gene, A pluripotency inducing factor may, but need not be, a pluripotency factor. Expression of an exogenously introduced pluripotency inducing factor may be transient, i.e., it may be needed during at least a portion of the reprogramming process in order to induce pluripotency and/or establish a stable pluripotent state but afterwards not required to maintain pluripotency. Examples of PIF of interest for reprogramming somatic cells to pluripotency in vitro are Ocl4, Nanog, Sox2, Lin28, Kit'd, c-hfyc, and any gene/protein that can substitute for one or more of these in a method of reprogramming somatic cells in vitro.

The term “Reprogramming factor” refers to a gene, RNA, or protein that promotes or contributes to cell reprogramming, e.g., in vitro.

The term “Hydrogel” refers to are three-dimensional, hydrophilic, polymeric networks capable of absorbing large amounts of water or biological fluids. Due to their high-water content, porosity, and soft consistency, they closely simulate natural living tissue.

The term “Mechanosensitive and stretch-activating ion channel (MSAIC)” refers to an ion channel that opens to allow passage of positively charged ions (i.e. cations) into and out of a cell in response to mechanical force or pressure being applied, e.g., to a cell expressing the channel. As used herein, the term also includes polypeptide components of mechanically-activated cation channels, e.g., subunits of a cation channel. In some embodiments, the mechanically-activated cation channels of the present invention are involved in sensory transduction, such as pain transduction, including but not limited to, cells such as neurons.

The term “Stem cells” refers to cells with the ability to divide for indefinite periods in culture and to give rise to specialized cells.

The term “Somatic cell” refers to any body cell other than gametes (egg or sperm).

The term “Embryonic stem cells” refers to primitive (undifferentiated) cells that are derived from preimplantation-stage embryos, are capable of dividing without differentiating for a prolonged period in culture, and are known to develop into cells and tissues of the three primary germ layers.

The term “Differentiation” refers to the process whereby an unspecialized embryonic cell acquires features of a specialized cell such as a heart, liver, or muscle cell. Differentiation is controlled by the interaction of a cell's genes with the physical and chemical conditions outside the cell, usually through signaling pathways involving proteins embedded in the cell surface.

The term “Pluripotent” refers to the state of a single cell that is capable of differentiating into all tissues of an organism, but not alone capable of sustaining full organismal development.

The term “Induced pluripotent stem cell” refers to a type of pluripotent stem cell, similar to an embryonic stem cell, formed by the introduction of certain embryonic genes into a somatic cell.

The term “Nuclear reprogramming” refers to describe changes in gene activity that are induced experimentally by introducing nuclei into a new cytoplasmic environment, a process by which the differentiation state of a cell is changed to that of another state.

The term “Pluripotent stem cell-like cell” refers to cells expressing PI factors.

The term “Yamanaka factor” refers to a set of genes, Oct3/4, Sox2, Klf4 and c-Myc, which are highly expressed in embryonic stem (ES) cells, and their over-expression can induce pluripotency in both mouse and human somatic cells, indicating that these factors regulate the developmental signaling network necessary for ES cell pluripotency.

The term “Cyclodextrins” refers to a family of cyclic oligosaccharides composed of α-(1,4) linked glucopyranose subunits. Cyclodextrins (CD) are useful molecular chelating agents. They possess a cage-like supramolecular structure, which is the same as the structures formed from cryptands, calixarenes, cyclophanes, spherands and crown ethers. These compounds having supramolecular structures carry out chemical reactions that involve intramolecular interactions where covalent bonds are not formed between interacting molecules, ions or radicals. The majority of all these reactions are of ‘host-guest’ type. Compared to all the supramolecular hosts mentioned above, cyclodextrins are most important. Because of their inclusion complex forming capability, the properties of the materials with which they complex can be modified significantly. As a result of molecular complexation phenomena CDs are widely used in many industrial products, technologies, and analytical methods. The negligible cytotoxic effects of CDs are an important attribute in applications such as drug carrier, food and flavors, cosmetics, packing, textiles, separation processes, environment protection, fermentation and catalysis.

### 5. SUMMARY OF THE INVENTION

This invention is based on the discovery that the inhibition specific for the mechanosensitive and stretch-activated ion channels (MSAICs) activates the expression of a set of PIFs in somatic cells (bone marrow cells), and subsequently rendered cells to acquire the pluripotent stem cell-like phenotypes. Further taught by the discovery was that somatic cells can be divided into two types after the inhibition of the MSAICs as 1) those that increase the expression of the PIFs within 16 hours (e.g., bone marrow cells) and 2) those that repressed the expression of the PIFs. When the second type cells such as fibroblasts were treated with MβCD, which reduces the cellular cholesterol contents, the expression of the PIFs was increased following MSAIC inhibition in 16 hours like the first type cells. Additional discovery showed that the second type cells, which reduces the expression of the PIFs, eventually increased the PIFs expression by 72 hours when the MSAIC inhibition continued. Thus, the sustained inhibition of the MSAICs activated the expression of the transcription factors in somatic cells to acquire the pluripotent stem cell-like phenotypes. The possibility for inducing the stem cell like phenotypes following the inhibition of MSAICs was investigated by the experimental conditions that attenuated the MSAICs signaling. Those tested were 1) to inhibit the MSAICs by GsMTX4, 2) to culture cells on the soft matrix made of polyacrylamide gel and 3) treat somatic cells with cholesterol depletion compound, such as MβCD. All approaches provided the proof of the principle to the invention that the attenuation of the MSAICs induced the repertoire of the transcription factors necessary for the stem cell-like phenotypes, and also showed the “re”-differentiated cells with novel phenotypes. The novel phenotype of the differentiated cells in the in vitro culture encompassed fat cells, bone forming cells, muscle cells, tendon forming cells, neurons, microglia, endothelial cells, erythrocyte progenitor cells etc. Finally, the application of the principle enabled the preparation of the somatic cell reprogramming kit, which includes the soft gel and the cholesterol depletion chemical. The invention generated stem cell-like cells expressing transcription factors including but not limited to Oct4, Nanog, Sox2 and c-Myc and subsequently differentiated into morphologically diverse somatic cells thus demonstrated that the invention is significantly beneficial for the regenerative medicine.

### 6. BRIEF DESCRIPTION OF THE DRAWINGS

FIG. 1 shows PI gene-specific activation by MSAIC inhibitor, GsMTX4, in bone marrow cells.

FIG. 2 shows the specific stiffness of the extra-cellular matrix triggered the induction of PI genes in bone marrow cells.

FIG. 3 shows the repression of PI gene expressions in spleen cells cultured 16 hours in the presence of GsMTX4.

FIG. 4 shows a bone marrow stroma cell line showing similar response to GsMTX4 as observed in fresh bone marrow cells.

FIG. 5 shows an embryonic fibroblast cell line showing similar response to GsMTX4 as observed in spleen cells.

FIG. 6 shows activation of PI genes in spleen cells on day 3 culture with GsMTX4.

FIG. 7 shows activation of PI genes in EF cells on day 3 culture with GsMTX4.

FIG. 8 shows that treatment of spleen cells with MβCD alters PI gene response against GsMTX4 similar to that of bone marrow cells.

FIGS. 9A-9B show microscopy of the spheres and dissolved sphere cells on the hydrogel. FIG. 1A shows spheres on the hydrogel after 16 hours incubation. FIG. 1B shows spheres dissolved to the round cell cluster on the hydrogel after 14 days.

FIGS. 10A-10C shows message analysis of PI genes of spheres on the hydrogel and cells on the dish bottom surface. FIG. 10A shows Oct4 gene expression; FIG. 10B shows Nanog gene expression; and FIG. 10C shows Sox2 gene expression.

FIG. 11 shows the nucleotide sequence of Oct4-specific RT-PCR fragment at 1000 bps (SEQ ID NO:1).

FIG. 12 shows the registered genomic nucleotide sequence of mouse Oct4 (SEQ ID NO:2).

FIG. 13 shows the nucleotide sequence of Nanog-specific RT-PCR fragment at 1000 bps (SEQ ID NO:3).

FIG. 14 shows the registered genomic nucleotide sequence of mouse Nanog (SEQ ID NO:4).

FIGS. 15A-15B shows the morphology and PI gene expression change overtime of MβCD-treated EF cells. FIG. 15A shows morphological change over time; FIG. 15B shows induction kinetics of PI genes.

FIG. 16 shows a microscopic image of EF cells.

FIG. 17A-17E show in vitro differentiated adipocyte-like cells derived from mouse EF cells, treated with MβCD and hydrogel. FIG. 17A shows brown adipocytes; FIG. 17B shows white adipocytes; FIGS. 17-C-E show oil-red-O-stained adipocytes.

FIGS. 18A-18C show in vitro differentiated osteoblasts derived from mouse EF cells treated with MβCD and hydrogel.

FIGS. 19A-19D show in vitro differentiated neuronal cell-like cells derived from mouse EF cells treated with MβCD and hydrogel. FIG. 19A shows cortical neurons; FIG. 19B shows astrocytes; FIG. 19C shows microglia; FIG. 19D shows oligodendrocytes.

FIG. 20 shows in vitro differentiated endothelial cell-like cells derived from mouse EF cells treated with MβCD and hydrogel.

FIGS. 21A-21F show in vitro differentiated cell clusters derived from mouse EF cells treated with MβCD and hydrogel. FIG. 21A shows astrocyte-like cells; FIG. 21B shows microglia-like cells; FIG. 21C shows clusters of non-classified cells. Cell types were labeled based on morphology.

FIGS. 22A-22C shows in vitro developed networks of neuronal cells derived from mouse EF cells treated with MβCD and hydrogel. Cell types were labeled based on morphology.

FIG. 23A-23D shows in vitro differentiated not-classified condensed cell clusters derived from mouse EF cells treated with MβCD and hydrogel.

FIG. 24 shows in vitro differentiated and matured adipocyte clusters derived from mouse EF cells treated with MβCD and hydrogel.

FIG. 25 in vitro differentiated myoblast-like cells derived from mouse EF cells treated with MβCD and hydrogel.

FIGS. 26A-26B show in vitro differentiated tendon cell/progenitor-like cells derived from mouse EF cells treated with MβCD and hydrogel. Cell types were labeled based on morphology.

FIG. 27A-27E show in vitro developed alignment of cells derived from mouse EF cells treated with MβCD and hydrogel.

FIG. 28 shows differentiation of cells from the sphere on polystyrene surface of tissue culture dish.

FIG. 29A-29C show protocols for reprogramming somatic cells to stem cell-like cells and differentiate into de novo phenotype cells.

FIG. 30 shows petri dishes casted with poly acrylamide gel and drop lid (arrow).

FIGS. 31A-31E show in vitro development of immature erythrocyte-like and megakaryocyte/platelet-like cells derived from human peripheral blood mononuclear cells treated with MβCD and hydrogel.

FIGS. 31A-31B show clusters of erythroblast-like cells; FIGS. 31C-31E show clusters of megakaryocyte/platelet-like cells.

FIG. 32 shows the somatic cell reprogramming culture setting instructed in the somatic cell reprogramming kit.

FIG. 33 shows a diagram of seeding somatic cells on the polyacrylamide gel.

### 7. DETAILED DESCRIPTION OF THE INVENTION

Provided herein are methods to produce pluripotent cells from non-pluripotent cells by 1) contacting non-pluripotent cells with one more cholesterol depletion agents, 2) contacting non-pluripotent cells with one or more MSAIC inhibitors, 3) culturing the cells on a soft matrix, or 4) a combination of any of the foregoing. When a combination is utilized, the steps can be performed sequentially or simultaneously. In particular embodiments, the method comprises 1) contacting non-pluripotent cells with one more cholesterol depletion agents and contacting non-pluripotent cells with one or more MSAIC inhibitors, 2) contacting non-pluripotent cells with one more cholesterol depletion agents and culturing the cells on a soft matrix, and 3) contacting non-pluripotent cells with one or more MSAIC inhibitors and culturing the cells on a soft matrix. In one embodiment, the cholesterol depletion agent is contacted with non-pluripotent cells prior to culturing the cells on a soft matrix. In a particular embodiment, the cells are contacted with the cholesterol depletion agent while the cells are in suspension in, for example a tube. In another embodiment, cells are contacted with the MSAIC inhibitor prior to culturing the cells on a soft matrix. In an embodiment, cells are contacted with the MSAIC inhibitor simultaneously with culturing the cells on a soft matrix. Particular cholesterol depletion agents and MSAIC inhibitors, as well as their concentrations and time of contact with cells are provided below. Similarly, the degree of softness of the matrix measured in kPa is provided below.

Various non-pluripotent cells can be induced according to the disclosed methods. Mammalian cells are preferred, including human cells. Cell types include human fibroblasts and human peripheral blood mononuclear cells. In an embodiment, the non-pluripotent cells are cells that are not genetically modified to express pluripotency inducing factors, such as Oct4, Nanog and Sox2. In an embodiment, the non-pluripotent cells that are not genetically modified to express pluripotency inducing factors are mammalian cells. In an embodiment, the non-pluripotent cells that are not genetically modified to express pluripotency inducing factors are human cells. In certain embodiments, the expression of one or more of the genes Oct4, Nanog and Sox2 is induced in the pluripotent stem cell relative to the non-pluripotent mammalian cell.

Various non-pluripotent cells can be induced according to the disclosed methods. Mammalian cells are preferred, including human cells. Cell types include human fibroblasts and human peripheral blood mononuclear cells. In certain embodiments, the expression of one or more of the genes Oct4, Nanog and Sox2 is induced in the pluripotent stem cell relative to the non-pluripotent mammalian cell.

MSAIC Inhibitors

One embodiment of the invention provides a method to induce pluripotency in non-pluripotent (somatic) cells by contacting the non-pluripotent cells with an MSAIC inhibitor. In certain embodiments, the MSAIC inhibitor activates in somatic cells the transcription of PIFs that includes but is not limited to Oct4, Nanog, Sox2 and c-Myc. MSAIC inhibitors are known, and include gadolinium, ruthenium red, and GsMTX4. In one embodiment, the MSAIC inhibitor is an inhibitor of the Piezo 1 stretch-activated channel. The preferred embodiment for the MSAIC inhibitor is GsMTX4. GsMTX4 is a water-soluble 34^{mer} peptide purified from a spider venom. Water-soluble inhibitors are preferred to those soluble only in in DMSO because expression of PIFs were found repressed by DMSO in the in vitro culture [Czysz et al., PLoS One 10 (2) (2015)]. Without being bound by theory, it is hypothesized that the inhibition of MSAICs with GsMTX4 stages the micro-environment on the polystyrene cell-culture surface (which possesses essentially infinite stretch force) simulates the environment of cells in contact with soft extracellular matrix.

In certain embodiments, cells are contacted with the MSAIC inhibitor at a concentration of at least 1 μM, between about 10 μM and about 1 μM, between 10 μM and 1 μM, between about 7 μM and about 3 μM, between 7 μM and 3 μM, about 5 μM or 5 μM. In particular embodiments, the MSAIC inhibitor is GsMTX4 at a concentration of between about 10 μM and about 1 μM, between 10 μM and 1 μM, between about 7 μM and about 3 μM, between 7 μM and 3 μM, about 5 μM or 5 μM. In a preferred embodiment, cells are contacted with GsMTX4 at a concentration of 5 μM.

In certain embodiments, cells are contacted with the MSAIC inhibitor for at least 12 hours, about 12 to about 96 hours, 12 to 20 hours, about 16 hours, 16 hours, 24 to 96 hours, 48 to 96 hours, about 72 hours, or for 72 hours. In one embodiment, cells are contacted with GsMTX4 at a concentration of 5 μM for 16 hours. In another embodiment, cells are contacted with GsMTX4 at a concentration of 5 μM for 72 hours.

In certain embodiments, cells are contacted with the MSAIC inhibitor at a temperature from about 4° C. to 42° C., a temperature from about 20° C. to 40° C., a temperature of about 37° C., a temperature of 37° C., a temperature of about 25° C., or a temperature of 25° C.

Cholesterol Depletion Agents

One embodiment of the invention provides a method to induce pluripotency in non-pluripotent (somatic) cells by contacting the non-pluripotent cells with a cholesterol depletion agent. In certain embodiments, the cholesterol depletion agent activates in somatic cells the transcription of PIFs that includes but is not limited to Oct4, Nanog, Sox2 and c-Myc. Cholesterol depletion agents include cylcodextrins, such as methyl-β-cyclodextrin (MβCD) hydroxypropyl-α-cyclodextrin (HPαCD), and hydroxypropyl-β-cyclodextrin (HPβCD).

Treatment with MSAIC inhibitors such as GsMTX4 divided somatic cells into two types, the first expressed PIFs within 16 hours, while the second expressed PIFs in a delayed manner after a period of the repression. One embodiment of the invention provides a method to change the second type of cell to the first type in vitro by treating with a cholesterol depletion agent. In one embodiment, the cholesterol depletion agent is one belonging to the cyclodextrin family. In preferred embodiment, the cellular cholesterol would be depleted by the treatment with MβCD (a cyclic oligosaccharide). After the depletion of the cellular cholesterol, treatment with GsMTX4 activated PI factors in the second type somatic cells within 16 hours. Thus, also provided herein are methods to convert second type somatic cells to first type somatic cells, which are readily reprogrammable into the pluripotent stem cell-like cells following the treatment with GsMTX4. The additional embodiment of the invention provides synergistic use of cellular cholesterol depletion and MSAIC inhibitor to reprogram somatic cells to pluripotent stem cell-like cells. The preferred embodiments to cause the synergy employ MβCD and GsMTX4.

In certain embodiments, cells are contacted with the cholesterol depletion agent at a concentration of at least 1 mM, between about 10 mM and about 1 mM, between 10 mM and 1 mM, between about 7 mM and about 3 mM, between 7 mM and 3 mM, about 5 mM or 5 mM. In particular embodiments, the cholesterol depletion agent is MβCD at a concentration of between about 10 mM and about 1 mM, between 10 mM and 1 mM, between about 7 mM and about 3 mM, between 7 mM and 3 mM, about 5 mM or 5 mM. In a preferred embodiment, cells are contacted with MβCD at a concentration of 5 mM.

In certain embodiments, cells are contacted with the cholesterol depletion agent for at least 12 hours, about 12 to about 96 hours, 12 to 20 hours, about 16 hours, 16 hours, 24 to 96 hours, 48 to 96 hours, about 72 hours, or for 72 hours. In one embodiment, cells are contacted with MβCD at a concentration of 5 mM for 20 minutes.

In certain embodiments, cells are contacted with the cholesterol depletion agent at a temperature from about 4° C. to 42° C., a temperature from about 20° C. to 40° C., a temperature of about 37° C., a temperature of 37° C., a temperature of about 25° C., or a temperature of 25° C.

Soft Extracellular Matrix

Another embodiment of the invention provides the method to induce pluripotency in somatic (non-pluripotent) cells by culturing cells on a soft extracellular matrix. In one embodiment, expression of PIFs in cells is induced culturing cells on soft extra cellular matrix. In preferred embodiment the soft extracellular matrix is made of hydrogel. The embodiment of the invention indicates that the hydrogel is polyacrylamide gel or a silicone gel. The softness of the polyacrylamide gel is tunable by altering the ratio of water, acrylamide, and bis-acrylamide. As shown in supra, the induction of the PIFs relies on the specific range of softness (indicated by pascals). Different type cells may require different softness of extra cellular matrix, which will be determinable by the person of ordinary skill in the art having benefit of the knowledge imparted by the teachings of this disclosure.

One embodiment of the invention provided the method to maintain in in vitro culture on a soft extracellular matrix the precursor pluripotent stem cells-like cells which express non-spliced messages of PIFs, Oct4 and Nanog.

In preferred embodiments the invention provided the examples of the softness at 7.4, 3.2 and 1.7 k pascal (kPa), in which 3.2 kPa is a preferred embodiment. In different embodiments, the extracellular matrix has a Young's elastic modulus of about 20 kPa or less, about 15 kPa or less, about 10 kPa or less, about 7.4 kPa or less, about 3.2 kPa or less, about 1.7 kPa or less, about 1 kPa or less, 20 kPa or less, 15 kPa or less, 10 kPa or less, 7.4 kPa or less, 3.2 kPa or less, 1.7 kPa or less, 1 kPa or less, 20 kPa, 15 kPa, 10 kPa, 7.4 kPa, 3.2 kPa, 1.7 kPa, 20 kPa, 15 kPa, 10 kPa, 7.4 kPa, 3.2 kPa, 1.7 kPa, 1 kPa.

### Differentiated Cells

One embodiment of the invention thus provides the method to differentiate types of somatic cells in the medium DMEM/F12 supplemented with 10% knock out serum replacement, lx non-essential amino acid, and 5×10⁻⁵M 2-Mercaptoethanol. Examples of differentiated cells include Oil red 0-positive white and brown adipocytes, neuronal cells, Alizarin positive osteoblast.

For example, brown adipocytes are in demand for the therapy of the obesity as they take up and consume large amounts of diverse nutrients simultaneously (e.g. glucose, lipids, amino acids) and can simultaneously engage both anabolic and catabolic metabolism [Payab, M. et al. Int J Obes (2020) https://doi.org/10.1038/s41366-020-0616-5]. The methods provided herein include methods differentiating pluripotent cells into various cell types, including adipocytes, neuronal cells, osteocytes, endothelial cells, erythrocytes, dendritic cells, platelets, lymphocytes, and myoblasts. In the case of neuronal cells, the neuronal cell type can include cortical neurons, astrocytes, microglia, and oligodendrocytes. Methods of inducing pluripotent cells to differentiate into different lineages are known, and can be used to differentiate the pluripotent cells created according to the teachings of the present disclosure.

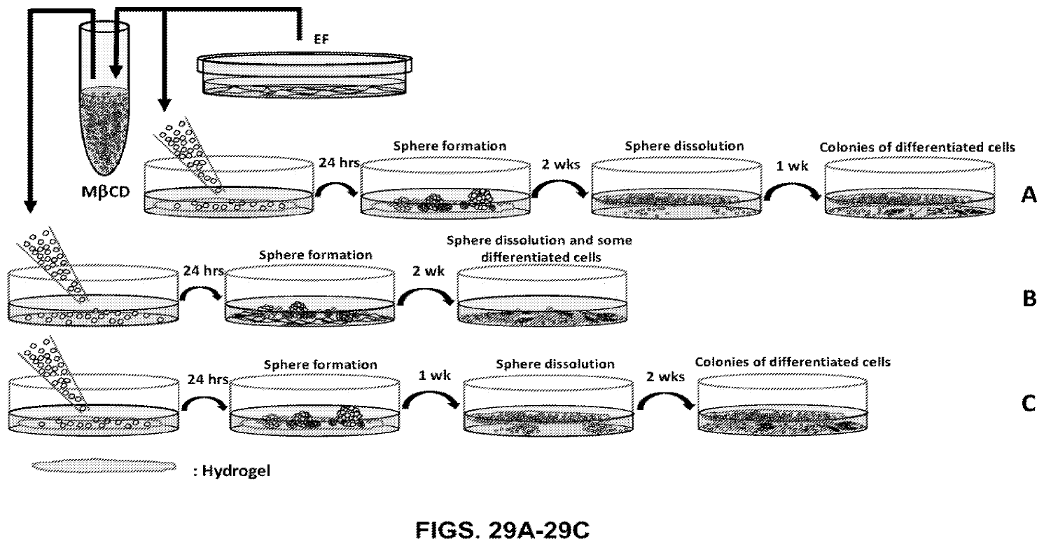

The embodiments of the invention supra provided the methods, in which the mechanosensitive and stretch-activated ion channels were attenuated with multiple methods combined (FIG. 29). For example, embryonic fibroblasts are treated with MβCD, then cultured on the soft hydrogels made of polyacrylamide. In another example, embryonic fibroblasts are treated with GsMTX4, then cultured on the soft hydrogels made of polyacrylamide. In an embodiment, the combined use of the cell-cholesterol depletion compound and soft extra-cellular matrix induces more frequent reprogramming and the increased appearance of cells possessing novel differentiated phenotypes. In additional embodiment, a somatic cell reprogramming kit is provided, of which the components comprise a cholesterol depletion agent, e.g., MβCD, and a low elasticity hydrogel, e.g., polyacrylamide, in the petri-dishes. In a particular embodiment, the polyacrylamide gel is air-dried following the casting in the 3.5 cm diameter petri dishes. The rehydration of the dried polyacrylamide with reprogramming medium prepares the low elastic cell culture matrix surface. The cholesterol-depletion solution is prepared by dissolving the fixed amount of a cholesterol depletion agent, e.g., MβCD powder, in the test tube with medium. In one embodiment, somatic cells contacted with the cholesterol depletion agent lower the contents of cholesterol and are cultured on low-elasticity polyacrylamide gels to generate stem cell-like cells.

Also provided are pluripotent cells produced according to the disclosed methods, pharmaceutical compositions comprising differentiated cells produced according to the disclosed methods, and reagents used in the methods, such as tissue culture media and cell culture containers. Cell culture containers include dishes, bottles, plates and multi-well plates.

All together the embodiment of the invention provided the novel methods and required tools to reprogram somatic cells to PSC-like cells, then re-differentiate them to various somatic cells useful for regenerative medicine. It is contemplated that the invention encompasses any aspect, or any combination of one or more aspects, of one or more of any of the embodiments presented herein.

### 8. EMBODIMENTS

Explicitly contemplated embodiments include the following:
1. A method of inducing a non-pluripotent mammalian cell into an induced pluripotent stem cell, the method comprising contacting the non-pluripotent mammalian cell with two or more of the following:
   a. one or more mechanosensitive and stretch-activated ion channel inhibitors in an amount sufficient to inhibit the mammalian cell ion channels;
   b. one or more cell cholesterol reducing agents in an amount sufficient to reduce the mammalian cell cholesterol level;
   c. a soft extracellular matrix having a Young's elastic modulus of 20 kPa or less.
2. The method of embodiment 1, wherein the non-pluripotent mammalian cell is not genetically modified to express pluripotency inducing factors.
3. The method of embodiment 1, wherein the mechanosensitive and stretch-activated ion channel inhibitor is selected from the group consisting of the L enantiomer of GsMTX4, the D enantiomer of GsMTX4, a peptide having a sequence at least 90% identical to the sequence of GsMTX4, or a mixture thereof
4. The method of embodiment 1, wherein the mechanosensitive and stretch-activated ion channel inhibitor is GsMTX4.
5. The method of embodiment 3, wherein the mechanosensitive and stretch-activated ion channel inhibitor is at a concentration between about 10 μM and about 1 μM.
6. The method of embodiment 3, wherein the mechanosensitive and stretch-activated ion channel inhibitor is at a concentration between 10 μM and 1 μM.
7. The method of embodiment 3, wherein the mechanosensitive and stretch-activated ion channel inhibitor is at a concentration between about 7 μM and about 3 μM.
8. The method of embodiment 3, wherein the mechanosensitive and stretch-activated ion channel inhibitor is at a concentration between 7 μM and 3 μM.
9. The method of embodiment 3, wherein the mechanosensitive and stretch-activated ion channel inhibitor is at a concentration of about 5 μM.
10. The method of embodiment 3, wherein the mechanosensitive and stretch-activated ion channel inhibitor is at a concentration of 5 μM.
11. The method of embodiment 3, wherein the cells are contacted with the mechanosensitive and stretch-activated ion channel inhibitor for about 12 to about 96 hours.
12. The method of embodiment 3, wherein the cells are contacted with the mechanosensitive and stretch-activated ion channel inhibitor for 12 to 20 hours.
13. The method of embodiment 3, wherein the cells are contacted with the mechanosensitive and stretch-activated ion channel inhibitor for about 16 hours.
14. The method of embodiment 3, wherein the cells are contacted with the mechanosensitive and stretch-activated ion channel inhibitor for 16 hours.
15. The method of embodiment 3, wherein the cells are contacted with the mechanosensitive and stretch-activated ion channel inhibitor for 24 to 96 hours.
16. The method of embodiment 3, wherein the cells are contacted with the mechanosensitive and stretch-activated ion channel inhibitor for 48 to 96 hours.
17. The method of embodiment 3, wherein the cells are contacted with the mechanosensitive and stretch-activated ion channel inhibitor for about 72 hours.
18. The method of embodiment 3, wherein the cells are contacted with the mechanosensitive and stretch-activated ion channel inhibitor for 72 hours.
19. The method of embodiment 1, wherein the cell cholesterol reducing agent is a cyclodextrin.
20. The method of embodiment 19, wherein the cyclodextrin is methyl-β-cyclodextrin.
21. The method of embodiment 20, wherein the cyclodextrin is at a concentration between about 10 mM and about 1 mM.
22. The method of embodiment 20, wherein the cyclodextrin is at a concentration between 10 mM and 1 mM.
23. The method of embodiment 20, wherein the cyclodextrin is at a concentration between about 7 mM and about 3 mM.
24. The method of embodiment 20, wherein the cyclodextrin is at a concentration between 7 mM and 3 mM.
25. The method of embodiment 20, wherein the cyclodextrin is at a concentration of about 5 mM.
26. The method of embodiment 20, wherein the cyclodextrin is at a concentration of 5 mM.
27. The method of embodiment 20, wherein the cells are contacted with the cyclodextrin for about 15 to about 60 minutes.
28. The method of embodiment 20, wherein the cells are contacted with the cyclodextrin for 15 to 60 minutes.
29. The method of embodiment 20, wherein the cells are contacted with the cyclodextrin for about 20 to about 40 minutes.
30. The method of embodiment 20, wherein the cells are contacted with the cyclodextrin for 20 to 40 minutes.
31. The method of embodiment 20, wherein the cells are contacted with the cyclodextrin for about 15 to about 30 minutes.
32. The method of embodiment 20, wherein the cells are contacted with the cyclodextrin for 20 to 30 minutes.
33. The method of embodiment 20, wherein the cells are contacted with the cyclodextrin for about 20 minutes.
34. The method of embodiment 20, wherein the cells are contacted with the cyclodextrin for 20 minutes.
35. The method of embodiment 20, wherein the cells are contacted with the cyclodextrin for about 30 minutes.
36. The method of embodiment 20, wherein the cells are contacted with the cyclodextrin for 30 minutes.
37. The method of embodiment 1, wherein the extracellular matrix has a Young's elastic modulus of about 15 kPa or less.
38. The method of embodiment 1, wherein the extracellular matrix has a Young's elastic modulus of about 10 kPa or less.
39. The method of embodiment 1, wherein the extracellular matrix has a Young's elastic modulus of about 7.4 kPa or less.
40. The method of embodiment 1, wherein the extracellular matrix has a Young's elastic modulus of about 3.2 kPa or less.
41. The method of embodiment 1, wherein the extracellular matrix has a Young's elastic modulus of about 1.7 kPa or less.
42. The method of embodiment 1, wherein the extracellular matrix has a Young's elastic modulus of about 1 kPa or less.
43. The method of embodiment 3, wherein the extracellular matrix has a Young's elastic modulus of about 15 kPa or less.
44. The method of embodiment 3, wherein the extracellular matrix has a Young's elastic modulus of about 10 kPa or less.
45. The method of embodiment 3, wherein the extracellular matrix has a Young's elastic modulus of about 7.4 kPa or less.
46. The method of embodiment 3, wherein the extracellular matrix has a Young's elastic modulus of about 3.2 kPa or less.
47. The method of embodiment 3, wherein the extracellular matrix has a Young's elastic modulus of about 1.7 kPa or less.
48. The method of embodiment 3, wherein the extracellular matrix has a Young's elastic modulus of about 1 kPa or less.
49. The method of embodiment 19, wherein the extracellular matrix has a Young's elastic modulus of about 15 kPa or less.
50. The method of embodiment 19, wherein the extracellular matrix has a Young's elastic modulus of about 10 kPa or less.
51. The method of embodiment 19, wherein the extracellular matrix has a Young's elastic modulus of about 7.4 kPa or less.
52. The method of embodiment 19, wherein the extracellular matrix has a Young's elastic modulus of about 3.2 kPa or less.
53. The method of embodiment 19, wherein the extracellular matrix has a Young's elastic modulus of about 1.7 kPa or less.
54. The method of embodiment 19, wherein the extracellular matrix has a Young's elastic modulus of about 1 kPa or less.
55. The method of embodiment 1, wherein the extracellular matrix is a hydrogel.
56. The method of embodiment 55, wherein the extracellular matrix is a polyacrylamide gel.
57. The method of embodiment 1, wherein the extracellular matrix is a silicone gel.
58. The method of embodiment 1, wherein the induced pluripotent stem cell is capable of differentiating into a cell type selected from the group consisting of adipocytes, neuronal cells, osteocytes, endothelial cells, erythrocytes, dendritic cells, platelets, lymphocytes, and myoblasts.
59. The method of embodiment 58, wherein the pluripotent stem cell is capable of differentiating into a neuronal cell, wherein the neuronal cell type is selected from the group consisting of cortical neurons, astrocytes, microglia, and oligodendrocytes.
60. The method of embodiment 1, wherein the non-pluripotent mammalian cell is a human cell selected from the group consisting of fibroblasts and peripheral blood mononuclear cells.
61. The method of embodiment 1, wherein the expression of one or more of the genes Oct4, Nanog and Sox2 is induced in the induced pluripotent stem cell relative to the non-pluripotent mammalian cell.
62. An embodiment comprising a method of inducing a non-pluripotent mammalian cell into an induced pluripotent stem cell, the method comprising contacting the non-pluripotent mammalian cell with two or more of the following:
   a. one or more mechanosensitive and stretch-activated ion channel inhibitors in an amount sufficient to inhibit the mammalian cell ion channels;
   b. one or more cell cholesterol reducing agents in an amount sufficient to reduce the mammalian cell cholesterol level;
   c. a soft extracellular matrix having a Young's elastic modulus of 20 kPa or less;
   wherein the non-pluripotent mammalian cell is not genetically modified to express pluripotency inducing factors;
   wherein the one or more mechanosensitive and stretch-activated ion channel inhibitors, if present, comprises GsMTX4 at a concentration of about 5 μM;
   wherein the one or more cholesterol reducing agents, if present, is methyl-β-cyclodextrin at a concentration of about 5 mM; and wherein the soft extracellular matrix, if present, has as Young's elastic modulus of about 7.5 kPa.
63. An embodiment comprising a method of inducing a non-pluripotent mammalian cell into an induced pluripotent stem cell, the method comprising contacting the non-pluripotent mammalian cell with two or more of the following:
   a. one or more mechanosensitive and stretch-activated ion channel inhibitors in an amount sufficient to inhibit the mammalian cell ion channels;
   b. one or more cell cholesterol reducing agents in an amount sufficient to reduce the mammalian cell cholesterol level;
   c. a soft extracellular matrix having a Young's elastic modulus of 20 kPa or less;
   wherein the non-pluripotent mammalian cell is not genetically modified to express pluripotency inducing factors;
   wherein the one or more mechanosensitive and stretch-activated ion channel inhibitors, if present, comprises GsMTX4 at a concentration of about 5 μM and the non-pluripotent mammalian cell is contacted with GsMTX4 for about 16 hours;
   wherein the one or more cholesterol reducing agents, if present, is methyl-β-cyclodextrin at a concentration of about 5 mM and the non-pluripotent mammalian cell is contacted with methyl-O-cyclodextrin for about 20 minutes; and wherein the soft extracellular matrix, if present, has as Young's elastic modulus of about 7.5 kPa.
64. The method of embodiment 62, wherein the induced pluripotent stem cell is capable of differentiating into a cell type selected from the group consisting of adipocytes, neuronal cells, osteocytes, endothelial cells, erythrocytes, dendritic cells, platelets, lymphocytes, and myoblasts.
65. The method of embodiment 62, wherein the pluripotent stem cell is capable of differentiating into a neuronal cell, wherein the neuronal cell type is selected from the group consisting of cortical neurons, astrocytes, microglia, and oligodendrocytes.
66. The method of embodiment 62, wherein the non-pluripotent mammalian cell is a human cell selected from the group consisting of fibroblasts and peripheral blood mononuclear cells.
67. The method of embodiment 62, wherein the expression of one or more of the genes Oct4, Nanog and Sox2 is induced in the induced pluripotent stem cell relative to the non-pluripotent mammalian cell.
68. The method of embodiment 63, wherein the induced pluripotent stem cell is capable of differentiating into a cell type selected from the group consisting of adipocytes, neuronal cells, osteocytes, endothelial cells, erythrocytes, dendritic cells, platelets, lymphocytes, and myoblasts.
69. The method of embodiment 63, wherein the pluripotent stem cell is capable of differentiating into a neuronal cell, wherein the neuronal cell type is selected from the group consisting of cortical neurons, astrocytes, microglia, and oligodendrocytes.
70. The method of embodiment 63, wherein the non-pluripotent mammalian cell is a human cell selected from the group consisting of fibroblasts and peripheral blood mononuclear cells.
71. The method of embodiment 63, wherein the expression of one or more of the genes Oct4, Nanog and Sox2 is induced in the induced pluripotent stem cell relative to the non-pluripotent mammalian cell.
72. A pharmaceutical composition comprising an isolated population of cells having a second non-pluripotent cell type, wherein the cells are obtained by a composition of converting animal cells from a first non-pluripotent cell type, and wherein the composition comprises inducing a non-pluripotent mammalian cell of a first cell type into an induced pluripotent stem cell by
   a. contacting the non-pluripotent mammalian cell with two or more of the following:
      i. one or more mechanosensitive and stretch-activated ion channel inhibitors in an amount sufficient to inhibit the mammalian cell ion channels;
      ii. one or more cell cholesterol reducing agents in an amount sufficient to reduce the mammalian cell cholesterol level;
      iii. a soft extracellular matrix having a Young's elastic modulus of 20 kPa or less, and
   b. inducing differentiation of the cells from step (a) into the second non-pluripotent cell type.
73. The composition of embodiment 72, wherein neither the first not the second non-pluripotent mammalian cell is genetically modified to express pluripotency inducing factors.
74. The composition of embodiment 72, wherein the mechanosensitive and stretch-activated ion channel inhibitor is selected from the group consisting of the L enantiomer of GsMTX4, the D enantiomer of GsMTX4, a peptide having a sequence at least 90% identical to the sequence of GsMTX4, or a mixture thereof.
75. The composition of embodiment 72, wherein the mechanosensitive and stretch-activated ion channel inhibitor is GsMTX4.
76. The composition of embodiment 74, wherein the mechanosensitive and stretch-activated ion channel inhibitor is at a concentration between about 10 μM and about 1 μM.
77. The composition of embodiment 74, wherein the mechanosensitive and stretch-activated ion channel inhibitor is at a concentration between 10 μM and 1 μM.
78. The composition of embodiment 74, wherein the mechanosensitive and stretch-activated ion channel inhibitor is at a concentration between about 7 μM and about 3 μM.
79. The composition of embodiment 74, wherein the mechanosensitive and stretch-activated ion channel inhibitor is at a concentration between 7 μM and 3 μM.
80. The composition of embodiment 74, wherein the mechanosensitive and stretch-activated ion channel inhibitor is at a concentration of about 5 μM.
81. The composition of embodiment 74, wherein the mechanosensitive and stretch-activated ion channel inhibitor is at a concentration of 5 μM.
82. The embodiment of claim 74, wherein the cells are contacted with the mechanosensitive and stretch-activated ion channel inhibitor for about 12 to about 96 hours.
83. The composition of embodiment 74, wherein the cells are contacted with the mechanosensitive and stretch-activated ion channel inhibitor for 12 to 20 hours.
84. The composition of embodiment 74, wherein the cells are contacted with the mechanosensitive and stretch-activated ion channel inhibitor for about 16 hours.
85. The composition of embodiment 74, wherein the cells are contacted with the mechanosensitive and stretch-activated ion channel inhibitor for 16 hours.
86. The composition of embodiment 74, wherein the cells are contacted with the mechanosensitive and stretch-activated ion channel inhibitor for 24 to 96 hours.
87. The composition of embodiment 74, wherein the cells are contacted with the mechanosensitive and stretch-activated ion channel inhibitor for 48 to 96 hours.
88. The composition of embodiment 74, wherein the cells are contacted with the mechanosensitive and stretch-activated ion channel inhibitor for about 72 hours.
89. The composition of embodiment 74, wherein the cells are contacted with the mechanosensitive and stretch-activated ion channel inhibitor for 72 hours.
90. The composition of embodiment 72, wherein the cell cholesterol reducing agent is a cyclodextrin.
91. The composition of embodiment 90, wherein the cyclodextrin is methyl-β-cyclodextrin.
92. The composition of embodiment 91, wherein the cyclodextrin is at a concentration between about 10 mM and about 1 mM.
93. The composition of embodiment 91, wherein the cyclodextrin is at a concentration between 10 mM and 1 mM.
94. The composition of embodiment 91, wherein the cyclodextrin is at a concentration between about 7 mM and about 3 mM.
95. The composition of embodiment 91, wherein the cyclodextrin is at a concentration between 7 mM and 3 mM.
96. The composition of embodiment 91, wherein the cyclodextrin is at a concentration of about 5 mM.
97. The composition of embodiment 91, wherein the cyclodextrin is at a concentration of 5 mM.
98. The composition of embodiment 91, wherein the cells are contacted with the cyclodextrin for about 15 to about 60 minutes.
99. The composition of embodiment 91, wherein the cells are contacted with the cyclodextrin for 15 to 60 minutes.
100. The composition of embodiment 91, wherein the cells are contacted with the cyclodextrin for about 20 to about 40 minutes.
101. The composition of embodiment 91, wherein the cells are contacted with the cyclodextrin for 20 to 40 minutes.
102. The composition of embodiment 91, wherein the cells are contacted with the cyclodextrin for about 15 to about 30 minutes.
103. The composition of embodiment 91, wherein the cells are contacted with the cyclodextrin for 20 to 30 minutes.
104. The composition of embodiment 91, wherein the cells are contacted with the cyclodextrin for about 20 minutes.
105. The composition of embodiment 91, wherein the cells are contacted with the cyclodextrin for 20 minutes.
106. The composition of embodiment 91, wherein the cells are contacted with the cyclodextrin for about 30 minutes.
107. The composition of embodiment 91, wherein the cells are contacted with the cyclodextrin for 30 minutes.
108. The composition of embodiment 72, wherein the extracellular matrix has a Young's elastic modulus of about 15 kPa or less.
109. The composition of embodiment 72, wherein the extracellular matrix has a Young's elastic modulus of about 10 kPa or less.
110. The composition of embodiment 72, wherein the extracellular matrix has a Young's elastic modulus of about 7.4 kPa or less.
111. The composition of embodiment 72, wherein the extracellular matrix has a Young's elastic modulus of about 3.2 kPa or less.
112. The composition of embodiment 72, wherein the extracellular matrix has a Young's elastic modulus of about 1.7 kPa or less.
113. The composition of embodiment 72, wherein the extracellular matrix has a Young's elastic modulus of about 1 kPa or less.
114. The composition of embodiment 74, wherein the extracellular matrix has a Young's elastic modulus of about 15 kPa or less.
115. The composition of embodiment 74, wherein the extracellular matrix has a Young's elastic modulus of about 10 kPa or less.
116. The composition of embodiment 74, wherein the extracellular matrix has a Young's elastic modulus of about 7.4 kPa or less.
117. The composition of embodiment 74, wherein the extracellular matrix has a Young's elastic modulus of about 3.2 kPa or less.
118. The composition of embodiment 74, wherein the extracellular matrix has a Young's elastic modulus of about 1.7 kPa or less.
119. The composition of embodiment 74, wherein the extracellular matrix has a Young's elastic modulus of about 1 kPa or less.
120. The composition of embodiment 90, wherein the extracellular matrix has a Young's elastic modulus of about 15 kPa or less.
121. The composition of embodiment 90, wherein the extracellular matrix has a Young's elastic modulus of about 10 kPa or less.
122. The composition of embodiment 90, wherein the extracellular matrix has a Young's elastic modulus of about 7.4 kPa or less.
123. The composition of embodiment 90, wherein the extracellular matrix has a Young's elastic modulus of about 3.2 kPa or less.
124. The composition of embodiment 90, wherein the extracellular matrix has a Young's elastic modulus of about 1.7 kPa or less.
125. The composition of embodiment 90, wherein the extracellular matrix has a Young's elastic modulus of about 1 kPa or less.
126. The composition of embodiment 72, wherein the extracellular matrix is a hydrogel.
127. The composition of embodiment 126, wherein the extracellular matrix is a polyacrylamide gel.
128. The composition of embodiment 72, wherein the extracellular matrix is a silicone gel.
129. The composition of embodiment 72, wherein the second non-pluripotent cell type is selected from the group consisting of adipocytes, neuronal cells, osteocytes, endothelial cells, erythrocytes, dendritic cells, platelets, lymphocytes, and myoblasts.
130. The composition of embodiment 129, wherein the second non-pluripotent cell type is a neuronal cell type selected from the group consisting of cortical neurons, astrocytes, microglia, and oligodendrocytes.
131. The composition of embodiment 72, wherein the first non-pluripotent cell type is a human cell selected from the group consisting of fibroblasts and peripheral blood mononuclear cells.
132. The composition of embodiment 72, wherein the expression of one or more of the genes Oct4, Nanog and Sox2 is induced in the first non-pluripotent cell type.
133. A pharmaceutical composition comprising an isolated population of cells having a second non-pluripotent cell type, wherein the cells are obtained by a composition of converting animal cells from a first non-pluripotent cell type, and wherein the composition comprises inducing a non-pluripotent mammalian cell of a first cell type into an induced pluripotent stem cell by
   a. contacting the non-pluripotent mammalian cell with two or more of the following:
      i. one or more mechanosensitive and stretch-activated ion channel inhibitors in an amount sufficient to inhibit the mammalian cell ion channels;
      ii. one or more cell cholesterol reducing agents in an amount sufficient to reduce the mammalian cell cholesterol level;
      iii. a soft extracellular matrix having a Young's elastic modulus of 20 kPa or less, and
   b. inducing differentiation of the cells from step (a) into the second non-pluripotent cell type;
   wherein the non-pluripotent mammalian cell is not genetically modified to express pluripotency inducing factors;
   wherein the one or more mechanosensitive and stretch-activated ion channel inhibitors, if present, comprises GsMTX4 at a concentration of about 5 μM;
   wherein the one or more cholesterol reducing agents, if present, is methyl-β-cyclodextrin at a concentration of about 5 mM; and wherein the soft extracellular matrix, if present, has as Young's elastic modulus of about 7.5 kPa.
134. The composition of embodiment 133, wherein the second non-pluripotent cell type is a cell type selected from the group consisting of adipocytes, neuronal cells, osteocytes, endothelial cells, erythrocytes, dendritic cells, platelets, lymphocytes, and myoblasts.
135. The composition of embodiment 133, wherein the second non-pluripotent cell type is a neuronal cell type, wherein the neuronal cell type is selected from the group consisting of cortical neurons, astrocytes, microglia, and oligodendrocytes.
136. The composition of embodiment 133, wherein the first non-pluripotent cell type is a human cell selected from the group consisting of fibroblasts and peripheral blood mononuclear cells.
137. The composition of embodiment 133, wherein the expression of one or more of the genes Oct4, Nanog and Sox2 is induced in the first non-pluripotent cell type.
138. A pharmaceutical composition comprising an isolated population of cells having a second non-pluripotent cell type, wherein the cells are obtained by a composition of converting animal cells from a first non-pluripotent cell type, and wherein the composition comprises inducing a non-pluripotent mammalian cell of a first cell type into an induced pluripotent stem cell by
   a. contacting the non-pluripotent mammalian cell with two or more of the following:
      i. one or more mechanosensitive and stretch-activated ion channel inhibitors in an amount sufficient to inhibit the mammalian cell ion channels;
      ii. one or more cell cholesterol reducing agents in an amount sufficient to reduce the mammalian cell cholesterol level;
      iii. a soft extracellular matrix having a Young's elastic modulus of 20 kPa or less, and
   b. inducing differentiation of the cells from step (a) into the second non-pluripotent cell type;
   wherein the non-pluripotent mammalian cell is not genetically modified to express pluripotency inducing factors;
   wherein the one or more mechanosensitive and stretch-activated ion channel inhibitors, if present, comprises GsMTX4 at a concentration of about 5 μM and the non-pluripotent mammalian cell is contacted with GsMTX4 for about 16 hours;
   wherein the one or more cholesterol reducing agents, if present, is methyl-β-cyclodextrin at a concentration of about 5 mM and the non-pluripotent mammalian cell is contacted with methyl-O-cyclodextrin for about 20 minutes; and
   wherein the soft extracellular matrix, if present, has as Young's elastic modulus of about 7.5 kPa.
139. The composition of embodiment 138, wherein the second non-pluripotent cell type is a cell type selected from the group consisting of adipocytes, neuronal cells, osteocytes, endothelial cells, erythrocytes, dendritic cells, platelets, lymphocytes, and myoblasts.
140. The composition of embodiment 138, wherein the second non-pluripotent cell type is a neuronal cell type, wherein the neuronal cell type is selected from the group consisting of cortical neurons, astrocytes, microglia, and oligodendrocytes.
141. The composition of embodiment 138, wherein the first non-pluripotent cell type is a human cell selected from the group consisting of fibroblasts and peripheral blood mononuclear cells.
142. The composition of embodiment 138, wherein the expression of one or more of the genes Oct4, Nanog and Sox2 is induced in the first non-pluripotent cell type.
143. A cell culture container comprising
   a. cell culture media,
   b. one or more mammalian cells treated with one or both of the following:
      i. one or more mechanosensitive and stretch-activated ion channel inhibitors in an amount sufficient to inhibit the mammalian cell ion channels;
      ii. one or more cell cholesterol reducing agents in an amount sufficient to reduce the mammalian cell cholesterol level; and
   c. a soft extracellular matrix having a Young's elastic modulus of 20 kPa or less.
144. The container of embodiment 143, wherein the mammalian cell is not genetically modified to express pluripotency inducing factors.
145. The container of embodiment 143, wherein the mechanosensitive and stretch-activated ion channel inhibitor is selected from the group consisting of the L enantiomer of GsMTX4, the D enantiomer of GsMTX4, a peptide having a sequence at least 90% identical to the sequence of GsMTX4, or a mixture thereof.
146. The container of embodiment 143, wherein the mechanosensitive and stretch-activated ion channel inhibitor is GsMTX4.
147. The container of embodiment 145, wherein the mechanosensitive and stretch-activated ion channel inhibitor is at a concentration between about 10 μM and about 1 μM.
148. The container of embodiment 145, wherein the mechanosensitive and stretch-activated ion channel inhibitor is at a concentration between 10 μM and 1 μM.
149. The container of embodiment 145, wherein the mechanosensitive and stretch-activated ion channel inhibitor is at a concentration between about 7 μM and about 3 μM.
150. The container of embodiment 145, wherein the mechanosensitive and stretch-activated ion channel inhibitor is at a concentration between 7 μM and 3 μM.
151. The container of embodiment 145, wherein the mechanosensitive and stretch-activated ion channel inhibitor is at a concentration of about 5 μM.
152. The container of embodiment 145, wherein the mechanosensitive and stretch-activated ion channel inhibitor is at a concentration of 5 μM.
153. The container of embodiment 143, wherein the cell cholesterol reducing agent is a cyclodextrin.
154. The container of embodiment 153, wherein the cyclodextrin is methyl-β-cyclodextrin.
155. The container of embodiment 154, wherein the cyclodextrin is at a concentration between about 10 mM and about 1 mM.
156. The container of embodiment 154, wherein the cyclodextrin is at a concentration between 10 mM and 1 mM.
157. The container of embodiment 154, wherein the cyclodextrin is at a concentration between about 7 mM and about 3 mM.
158. The container of embodiment 154, wherein the cyclodextrin is at a concentration between 7 mM and 3 mM.
159. The container of embodiment 154, wherein the cyclodextrin is at a concentration of about 5 mM.
160. The container of embodiment 154, wherein the cyclodextrin is at a concentration of 5 mM.
161. The container of embodiment 143, wherein the extracellular matrix has a Young's elastic modulus of about 15 kPa or less.
162. The container of embodiment 143, wherein the extracellular matrix has a Young's elastic modulus of about 10 kPa or less.
163. The container of embodiment 143, wherein the extracellular matrix has a Young's elastic modulus of about 7.4 kPa or less.
164. The container of embodiment 143, wherein the extracellular matrix has a Young's elastic modulus of about 3.2 kPa or less.
165. The container of embodiment 143, wherein the extracellular matrix has a Young's elastic modulus of about 1.7 kPa or less.
166. The container of embodiment 143, wherein the extracellular matrix has a Young's elastic modulus of about 1 kPa or less.
167. The container of embodiment 145, wherein the extracellular matrix has a Young's elastic modulus of about 15 kPa or less.
168. The container of embodiment 145, wherein the extracellular matrix has a Young's elastic modulus of about 10 kPa or less.
169. The container of embodiment 145, wherein the extracellular matrix has a Young's elastic modulus of about 7.4 kPa or less.
170. The container of embodiment 145, wherein the extracellular matrix has a Young's elastic modulus of about 3.2 kPa or less.
171. The container of embodiment 145, wherein the extracellular matrix has a Young's elastic modulus of about 1.7 kPa or less.
172. The container of embodiment 145, wherein the extracellular matrix has a Young's elastic modulus of about 1 kPa or less.
173. The container of embodiment 153, wherein the extracellular matrix has a Young's elastic modulus of about 15 kPa or less.
174. The container of embodiment 153, wherein the extracellular matrix has a Young's elastic modulus of about 10 kPa or less.
175. The container of embodiment 153, wherein the extracellular matrix has a Young's elastic modulus of about 7.4 kPa or less.
176. The container of embodiment 153, wherein the extracellular matrix has a Young's elastic modulus of about 3.2 kPa or less.
177. The container of embodiment 153, wherein the extracellular matrix has a Young's elastic modulus of about 1.7 kPa or less.
178. The container of embodiment 153, wherein the extracellular matrix has a Young's elastic modulus of about 1 kPa or less.
179. The container of embodiment 143, wherein the extracellular matrix is a hydrogel.
180. The container of embodiment 179, wherein the extracellular matrix is a polyacrylamide gel.
181. The container of embodiment 143, wherein the extracellular matrix is a silicone gel.
182. A cell culture container comprising
   a. cell culture media,
   b. one or more mammalian cells treated with one or both of the following:
      i. one or more mechanosensitive and stretch-activated ion channel inhibitors in an amount sufficient to inhibit the mammalian cell ion channels;
      ii. one or more cell cholesterol reducing agents in an amount sufficient to reduce the mammalian cell cholesterol level; and
   c. a soft extracellular matrix having a Young's elastic modulus of 20 kPa or less:
   wherein the non-pluripotent mammalian cell is not genetically modified to express pluripotency inducing factors;
   wherein the one or more mechanosensitive and stretch-activated ion channel inhibitors, if present, comprises GsMTX4 at a concentration of about 5 μM;
   wherein the one or more cholesterol reducing agents, if present, is methyl-β-cyclodextrin at a concentration of about 5 mM; and
   wherein the soft extracellular matrix, if present, has a Young's elastic modulus of about 7.5 kPa.
183. A method of increasing expression of an endogenous pluripotency inducing transcription factor in a somatic cell, comprising modifying the signaling of cell membrane receptors.
184. The method of embodiment 183, wherein the somatic cell is a fibroblast.
185. The method of embodiment 183, wherein the pluripotency inducing transcription factor is selected from Oct-4, Sox-2, Nanog and c-Myc.
186. The method of embodiment 183, wherein the cell membrane receptor is mechanosensitive and/or stretch-activated ion channel
187. The method of embodiment 183, wherein the cell membrane receptor signaling is modified by contacting the cell with a mechanosensitive and/or stretch-activated ion channel-specific inhibitor.
188. The method of embodiment 187, wherein the inhibitor is GsMTX4.
189. The method of embodiment 183, wherein the cell membrane receptor signaling is modified by depletion of at least one cellular lipid.
190. The method of embodiment 189, wherein the at least one cellular lipid is cholesterol.
191. The method of embodiment 189, wherein depletion of the cellular lipid occurs by contacting the cell with a molecule of the cyclodextrin family.
192. The method of embodiment 191, wherein the molecule is methyl-beta-cyclodextrin.
193. The method of embodiment 183, wherein the cell membrane receptor signaling is modified by culturing the cells in a low elastic extra cellular matrix.
194. The method of embodiment 193, wherein the matrix is a polyacrylamide gel.
195. The method of embodiment 194, wherein the polyacrylamide gel elasticity is lower than 7.4 k pascal.
196. The method of embodiment 183, wherein the cell membrane receptor signaling is modified by
   a. contacting the cell with a mechanosensitive and/or stretch-activated ion channel-specific inhibitor;
   b. depletion of at least one cellular lipid; and
   c. culturing the cells in a low elastic extra cellular matrix.
197. A cell produced by the method of embodiment 183, which expresses endogenous pluripotency transcription factors.
198. A cell produced by the method of embodiment 183, which expresses pluripotency inducing transcription factors.
199. A differentiated cell, which is derived from the cell of embodiment 198.
200. A kit for performing the method of embodiment 183.
201. The kit of embodiment 200, comprising a cholesterol depletion compound and a low elastic extra cellular matrix.
202. The kit of embodiment 200, comprising methyl-β-cyclodextrin and dried polyacrylamide gel.

### 9. EXAMPLES

The following Examples, which highlight certain features and properties of the exemplary embodiments of the invention described herein are provided for purposes of illustration, and not limitation.

### 9.1. Example 1

9.1.1. Materials and Methods

Mice, Cells and Antibodies

C57BL/6 male mice (4-6 weeks old) were purchased from Jackson Laboratory. Bone marrow cells and spleen cells were obtained from 4-6-week-old male C57BL/6 mice. Cells in single cell suspension were red cell-depleted before use. Bone marrow stromal cells were cultured and passaged in T75 culture flask as it was described [Torino et al., Bio-protocol 4: e1031 (2014)]. The embryonic fibroblast was prepared from 13-day fetus of C57BL/6 as described previously [Qiu et al., Bio-protocol 6: e1859 (2016)]. Cells used for the reprogramming were those cultured for 2 to 4 passage periods.

MSAIC Inhibition Assay with GsMTX4

Cells were cultured in 6 well plates (10⁷ cells/well) in triplicate per group. GsMTX4 dissolved in PBS was added to the concentration 5 μM in the experimental group. Sixteen hours later, cells were harvested by scraper for spleen cells and bone marrow cells, pelleted for total RNA-extraction with RNeasy (Qiagen) or Trizol (Invitrogen). For EF cells and BM stromal cells, cell culture medium was drained and directly resuspended to the cell lysis buffer in the culture wells. Extracted RNAs were subjected to cDNA synthesis (Applied Bioscience) and analyzed for the messages specific for mouse PI genes and control genes by real time PCR thereto cycler (Bio-Rad, CFX384). Primers used were: Oct4 (5′: CTACAGTCCCAGGACATGAA (SEQ ID NO:5), 3′: TGGTCTCCAGACTCCACCTC (SEQ ID NO:6), Sox2 (5′: ATGATGGAGACGGAGCTGAA (SEQ ID NO:7), 3′: TTGCTGATCTCCGAGTTGTG (SEQ ID NO:8), Nanog (5′: AAGTACCTCAGCCTCCAGCA (SEQ ID NO:90), 3′: GCTTGCACTTCATCCTTTGG (SEQ ID NO:10), CFBP/α (5′: CGACTTCTACGAGGTGGAGC (SEQ ID NO:11), 3′: TCGATGTAGGCCGCTGATGTC (SEQ ID NO:12), c-Myc (5′: CACCATGCCCCTCAACGTGA (SEQ ID NO:13), 3′: TTATGCACCAGAGTTTCG (SEQ ID NO:14), RUNX1 (5′: CGTATCCCCGTAGATGGCAG (SEQ ID NO:15), 3′: GCCAGGGTGGTCAGCTAGTA (SEQ ID NO:16), PU1 (5′: AGAGCATACCAACGTCCAATGC (SEQ ID NO:17), 3′: GTGCGGAGAAATCCCAGTAGTG (SEQ ID NO:18), IRF8 (5′: CGTGGAAGACGAGGTTACGCTG (SEQ ID NO:19), 3′: GCTGAATGGTGTGTGTCATAGGC (SEQ ID NO:20), FOXP1 (5′: ATCCCAGAACGGGTCCAGCGGTGGCAACCAC (SEQ ID NO:21), 3′: GATCTGCTGCATTTGTTGAGGAGTGATAAC (SEQ ID NO:22), KLF4 (5′: GGTGCAGCTTGCAGCAGTAA (SEQ ID NO:23), 3′: AAAGTCTAGGTCCAGGAGGTCGTT (SEQ ID NO:24), Actin (5′: GTGACGAGGCCCAGAGCAAGAG (SEQ ID NO:25), 3′: AGGGGCCGGACTCATCGTACTC (SEQ ID NO:26), GAPDH 5′: CATCACCATCTTCCAGGAGCG (SEQ ID NO:27), 3′: ACGGACACATTGGGGGTAGG (SEQ ID NO:28). The value of each Cq (Ct) were applied to the formula by which the message level of each specific gene (shown by the index value) was estimated in comparison to that of Actin or GAPDH housekeeping genes.

Low Elasticity Tissue Culture Plates and Hydrogel Casting in Culture Plates

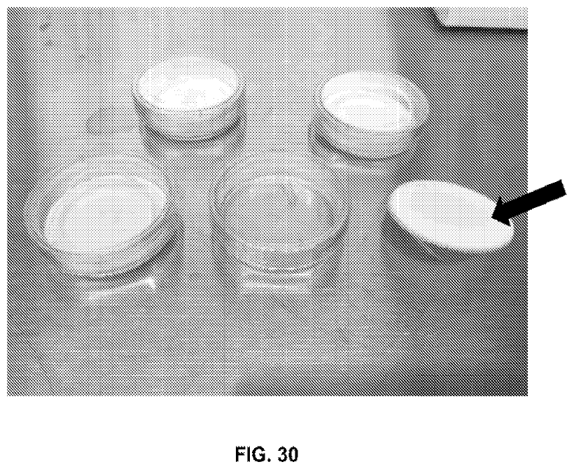

Tissue culture plates with defined elasticity surface (0.2, 4, 50 kpa) were purchased from Matrigen. To cast polyacrylamide gel in petri-dishes, 3.5 cm petri-dish was used. Each dish was loaded with 330 μL of polyacrylamide gel cocktail and promptly the size-matched drop lid (polypropylene) was placed on the top (FIG. 30). Three different elasticities of the polyacrylamide gel were prepared in the study. The ratio of [acrylamide (40%): bis-acrylamide (2%): water] was determined according from the published data [Tse and Engler, Curr. Protoc. Cell Biol. Chapt. 10 Unit 10.16 (2010)]. Those were (75:112.5:812.5) for 1.7 kpa, (100:150:750) for 3.2 kpa and (250:30:720) for 7.4 kpa. The slice of the 50 ml conical centrifuge tube caps were used for the drop caps (arrow-pointed in FIG. 30).

Ninety minutes later drop lids were carefully removed and the gels in the dishes were washed 5 times with 3 ml of sterile water. Each aspiration of the water from the dish was gently performed with 1 ml micro pipettor. During the washing, casted acrylamide gel come off the bottom. Lastly gels were saturated with DMEM/F12 medium for two hours before use. Every component for polyacrylamide gel except TEMED was filter sterilized. Drop lids were sterilized with 70% Et-OH and dried in the tissue culture hood. Whole process was performed in the tissue culture hood.

Treatment of Cells with MβCD and Reprogramming Culture

Cells were suspended in DMEM supplemented with 5 mM MβCD at the concentration of 2×10⁶/ml in 15 ml centrifuge tube. Cell suspension was incubated for 30 min at 37° C. with every 5 min gentle agitations. Washed once with 37° C. warmed DMEM/F12 and resuspended in small volume complete medium (30 μl/2×10⁶ cells) [DMEM/F12 (Corning), 20% knockout serum replacement (Gibco), 1% nonessential amino acids (Gibco), 5×10⁻⁵M 2-Mercaptethanol]. To initiate reprogramming culture on hydrogel, petri dish was added with 2.5 ml complete medium, then 30 μl of cell suspension was loaded on the hydrogel. Multiple dishes were housed in a 15 cm diameter petri dish with a water filled un-lid 6 cm petri dish. Cells on the gel and on the dish-bottom surface were monitored daily and the remarkable phenotype cells were recorded with EVOS cell monitoring system (Thermofisher). Various phenotype cells were characterized by the assays for the markers specific for the cell lineages (e.g., Oil red 0 for the adipocytes and alizarin for calcium deposited cells) as commonly available methods.

RT-PCR Assay for the Expression of PI Genes in the Reprogramming Cultures of EF Cells

Cells derived from the MβCD-treated and hydrogel-exposed culture in petri dish were subjected to the extraction of total RNA by Trizol. cDNAs were prepared as described above and the regular PCR was performed (35 cycle) using PCR master mix (Biotool) and the primers listed above for the real time PCR assays. The PCR products were resolved using 1.5% agarose gel, Ethidium Bromide-stained and the results were acquired by Gel Documentation system (Bio-Rad).

### 9.2. Example 2

Nuclear Reprogramming of Human Peripheral Blood Mononuclear Cells (PBMC)

Human PBMC is an attractive source of somatic cells for reprogramming to stem cells by the available iPSC technology. The sampling of the peripheral blood is a common practice in the medicine and possible to obtain at the individual specific fashion. The purification of the PBMC is also a regular method by Ficol gradient centrifugation. Previously, the standard iPSC technology by using Lentivirus resulted in the establishments of the set of iPSCs [Simara, Pavel et al. Stem cells and development vol. 27, 10-(2018), U.S. Pat. No. 9,447,382B2]. To investigate if the methods of the current invention can apply to reprogram human PBMC into the stem cell-like cells, and also differentiate into the de novo phenotypes in vitro, experiments were conducted in which cell were treated with MβCD and then cultured on the soft polyacrylamide gel as described infula.

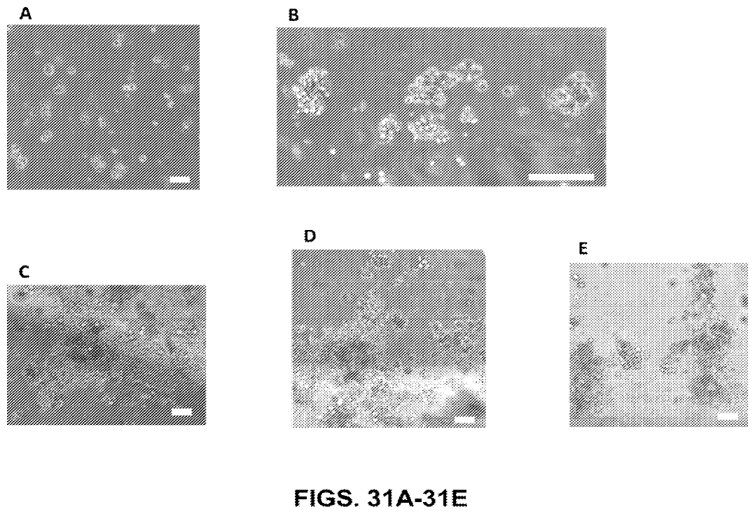

Human peripheral blood was corrected into the heparinized tube from left forearm vein. The blood mononuclear cells were separated using the Ficoll-Hypaque technique (density, 1.077; Pharmacia Biotech, Uppsala, Sweden). Cells were twice repeatedly 37° C. incubated for 20 min with 5 mM MβCD in DMEM, pelleted by centrifugation at 900 g at room temperature. Washed once with 37° C. warmed DMEM/F12 and resuspended in small volume complete medium (30 μl/2×10⁶ cells) [DMEM/F12 (Corning), 20% knockout serum replacement (Gibco), 1% nonessential amino acids (Gibco), 5×10⁻⁵M 2-Mercaptethanol]. Cells in the culture petri-dishes were monitored daily by microscopy and novel phenotype cells were recorded. As shown in FIGS. 31A-31D, we observed novel-phenotype cells, resembling to erythrocytes, erythrocyte progenitor cells, dendritic cell like cell, lymphocyte like small cells and platelet like small particles. Those cells were recognized after a week of the culture and the number of the immature erythrocyte-like cell were increased as the culture period was extended longer than 2 weeks. The control culture, which was not treated with MβCD nor soft hydrogel showed large number of cell death and no similar landscape was observed by the microscopy.

FIGS. 31A-31D show in vitro development of immature erythrocyte-like and megakaryocyte/platelet-like cells derived from human peripheral blood mononuclear cells treated with MβCD and hydrogel. FIGS. 31A-31B show clusters of erythroblast-like cells, and FIGS. 31C-31E show clusters of megakaryocyte/platelet-like cells.

Importantly, the erythrocytes production is known to be strictly limited to originated from the bone marrow cells and not from the peripheral blood mononuclear cells [Dzierzak and Sjaak, Cold Spring Harbor perspectives in medicine vol. 3,4 a011601. 1 Apr. 2013, doi:10.1101/cshperspect.a011601]. Thus, the invention provided the unique and valuable opportunity to generate cells to meet the demand, to transfuse blood cells to the anemic as well as the lymphocytopenia.

### 9.3. Example 3

Somatic Cell Reprogramming Kit

The current discovery taught that somatic cells are reprogrammable to pluripotent stem cell-like cells after the attenuation of the mechanical stress. In this context, the in vitro culture on the low pascal (eg., 3.2 kpa) acrylamide gel surface transform fibroblastic cells to pluripotent stem cell-like cells. Additionally, the cellular cholesterol depletion with MβCD reprogram fibroblastic cells to pluripotent stem cell-like cells. Potentially, those techniques could generate novel platforms to reprogram somatic cells to stem cells and the subsequent differentiation to different type of cells. A kit that enables standard skilled person to reprogram somatic cells comprises a 3.5 cm diameter petri dish, in which soft polyacrylamide gel is casted and dried, and an aliquot of MβCD powder in a tube. The following describes the “Materials, Methods, and Procedures” associates with the “Somatic cell reprogramming kit”

9.3.1. Materials and Methods

Tubes containing reprogramming reagents (MβCD). Each tube for one cell type reprogramming.

A set 35 mm petri-dishes containing dried stem cell matrix (acrylamide gel). Each petri dish has dried polyacrylamide gel with different elasticity after the rehydration as one has the 1.7 kpa, second, 3.2 kpa, and the third has 7.4 kpa. The different softness of the matrix in each dish, thus could be selected for the optimum reprogramming and the subsequent re-differentiation for the specific somatic cell of interest.
DMEM with antibiotics
Complete medium: DMEM/F-12 with antibiotics, Knockout serum (20%), Nonessential amino acid (1×), 2-Mercaptoethanol at 5×10⁻⁵M.
Petri dishes (14 cm diameter; 6 cm diameter)
Disposable 10 ml syringe
Syringe filter (0.22 μm)
Cell imaging system to keep records, e.g., EVOS cell imaging system (ThermoFisher).

Daily Protocol

### Day 1

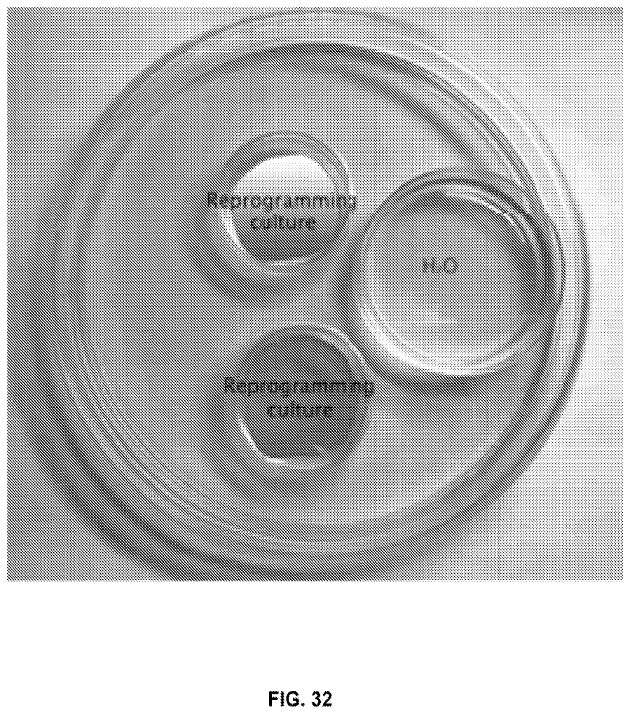

In the tissue culture hood, place petri-dishes in a 14 cm petri-dish, which houses a clean water filled 5.5 cm petri-dish without lid. Add 3 ml of the complete medium to 3.5 cm petri-dishes with stem cell matrix (dried polyacrylamide gel).
See FIG. 32 for example.

### Day 2

Monitor the petri-dishes for any contamination and the presence of the reconstituted stem cell matrix.

### Day 3

Monitor the petri-dishes for any contamination.
Prepare the reprogramming solution right before the use. Dissolve the reprogramming reagents in the tube to 5 ml DMEM. Voltex well to mix/dissolve completely and sterilize by the syringe filter.

### Warm at 37° C.

Harvest embryonic fibroblast cells (3-5 million cells) to 15 ml conical tube.
Wash once with DMEM.

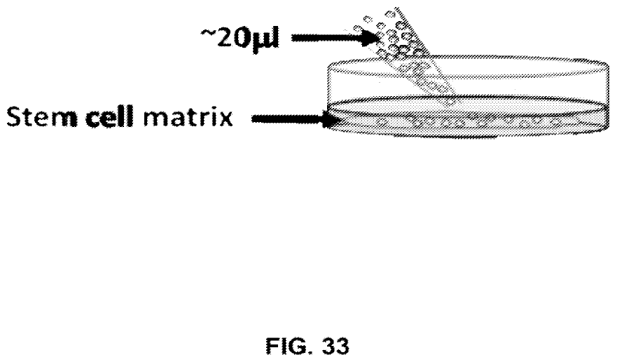

Resuspend cell pellets to the half volume of the reprogramming MβCD solution (˜2.5 ml), incubate at 37° C. for 20 min with mixing every 5 min. Spin and pellet, remove the supernatant then resuspend again to the remaining half (˜2.5 ml) of the reprogramming solution, continue additional 20 min 37° C. incubation with every 5 min mixing. Spin down (no need to wash) and resuspend the pellet to 20-30 μl complete medium. With 200 μl pipetman, gently seed cells on the stem cell matrix in the 35 mm petri-dish. Seed ˜20 μl on the matrix per petri-dish (FIG. 33). Wait 20 min till cells settle down on the matrix in the tissue culture hood. Gently transfer whole petri-dish combo into the 37° C. CO₂ incubator to start reprogramming/differentiation culture.

### Day 7

Add βFGF (10 ng/ml)

### Day 8 and Beyond

Monitor cells on the matrix and the bottom surface of the culture.

### 9.4. Discussion

The investigation was conducted to test if the signaling of MSAIC in bone marrow cells could activate the expression of pluripotency inducing (PI) genes. To modify the signaling of MSAICs the employed was GsMTX4 [Gnanasambandam et al., Biophys. J. 112:31 (2017)], which possesses the specific functional blocking activity against MSAICs [Park et al., PAIN, 137:208 (2008)]. GsMTX4 is a water soluble 34^{mer} peptide purified from the venom of the Theraphosidae family spider (Tarantula) that was previously used to increase the mechanical threshold of sensory neurons for touch, pressure, proprioception, and pain [Bowman et al., Toxicon, 49:249 (2007)]. To characterize the PI gene expression, the water soluble GsMTX4 was useful because some other MSAIC inhibitors (e.g., HC 067047 [Everaerts et al., Proc. Natl. Acad. Sci. USA 107:19084 (2010)]) are only soluble in DMSO or alcohols, which were by themselves known to alter the expression of PI genes [Czysz et al., PLoS One, 10 (2) (2015); Ogony et al., Stem Cells Dev., 22: 2196 (2013)].

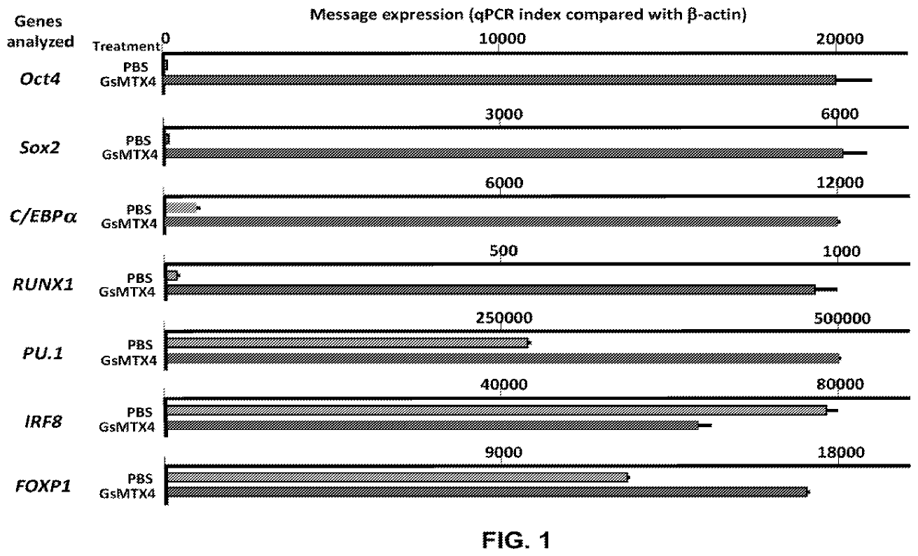

In experiments, mouse bone marrow cells were in vitro 37° C. cultured 16 hours in the presence of 5 μM GsMTX4. Cells were then assayed for the PI genes' messages as listed in the left of the FIG. 1. In this cell culture, cells were exposed to hard polystyrene surface, which possesses essentially infinite stretch force. The presence of GsMTX4 in the culture was expected to prevent cells from sensing the strong stretch stress by MSAICs. Strikingly, the cells cultured in the presence of GsMTX4 expressed greater than 100 times of Oct4 message compared with the control cells (FIG. 1). Also activated prominently in the cells were Sox2, C/EBPα and RUNX1, which were reportedly involved in the hematopoietic stem cell phenotype [Hasemann et al., PLoS Genet. 10(1): e1004079 (2014); North et al., Stem Cells. 22:158 (2004)]. Thus, the data showed that the treatment with GsMTX4 greatly activated the specific gene expression important to the stem cell phenotype.

The fresh bone marrow cells were cultured for 16 hours in 6 well plates with GsMTX4 (5 μM) in DMEM supplemented with 10% FBS. The messages specific for the genes listed in the left were real time PCR-characterized and presented as the relative index in comparison with the value of β-actin message. Each experimental value shown was the average and the standard deviation of triplicate samples. The results shown represent three experiments with similar results.

When RNAs were assayed for the expression of non-PI genes, IRF8 and FOXP1, the expression change was small and no compare to that of PI genes. Therefore, GsMTX4 enhanced the PI gene expression in the gene-specific manner. The study showed that the stretch insults detected by the MSAICs, sensitively/dominantly suppressed the expression of PI genes in bone marrow cells. It may be construed that the stiffer extra-cellular matrix-stimulated MSAIC signaling promotes the stem cell differentiation by reducing the pluripotent differentiation potentials.

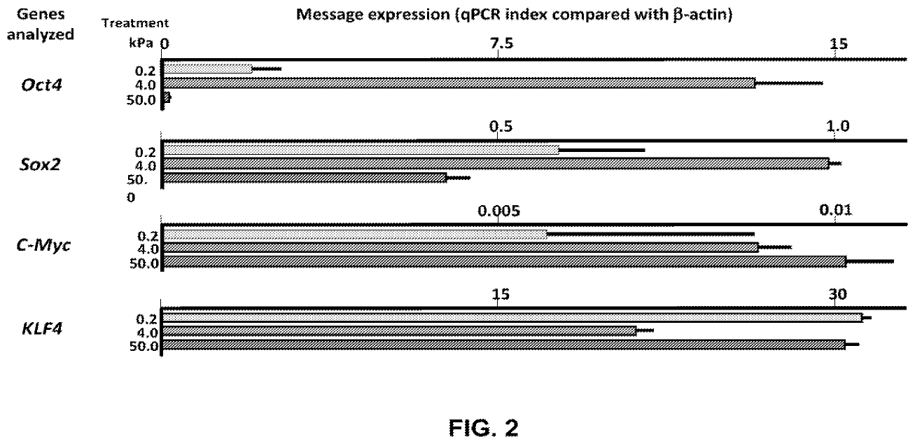

The GsMTX4-mediated inhibition of MSAICs is assumed to render the micro-environment, in which cells were in contact with the soft extracellular matrix. In this context, to obtain the insights in depth, the investigated was the response of bone marrow cells cultured in various stiffness tissue culture wells. The stiffness chosen were 0.2 k pascal, 4 k pascal and 50 k pascal. Cells were cultured 16 hours in the wells and assayed for the PI genes-expression as described above. Significantly, the prominent expression of the PI genes was observed at the specific stiffness as 4 k pascal induced the highest and 0.2 or 50 k pascal showed lower levels of induction (FIG. 2).

Bone marrow cells were cultured in the 6 well plates coated with the specific stiffness matrix as listed in the left. The messages of the genes listed left were qPCR-characterized as described in the FIG. 1. The results indicated that the activation of PI genes depended on the specific range of the extra-cellular matrix stiffness as the excessively soft or stiff did not lead to the greatly increased expression of PI genes.

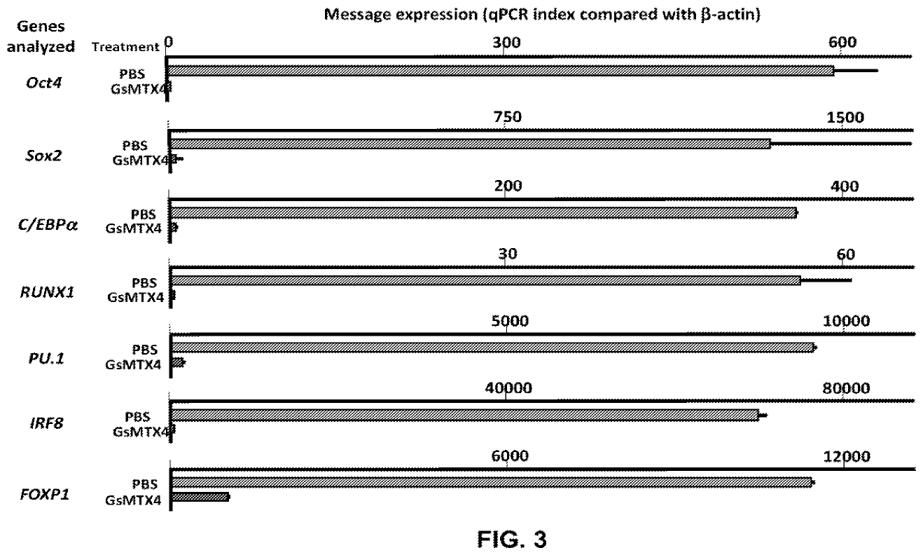

The regulation of PI genes in spleen cells by GsMTX4 contradicts to that observed in bone marrow cells. When spleen cells were cultured 16 hours in the presence of GsMTX4 the activation of PI genes was not observed, instead, although the expression level was low, the PI genes expression was consistently repressed (FIG. 3). The spleen cells were cultured in 6 well plates as described in FIG. 1. Gene expressions listed in the left were evaluated by the same methods as shown in FIG. 1.

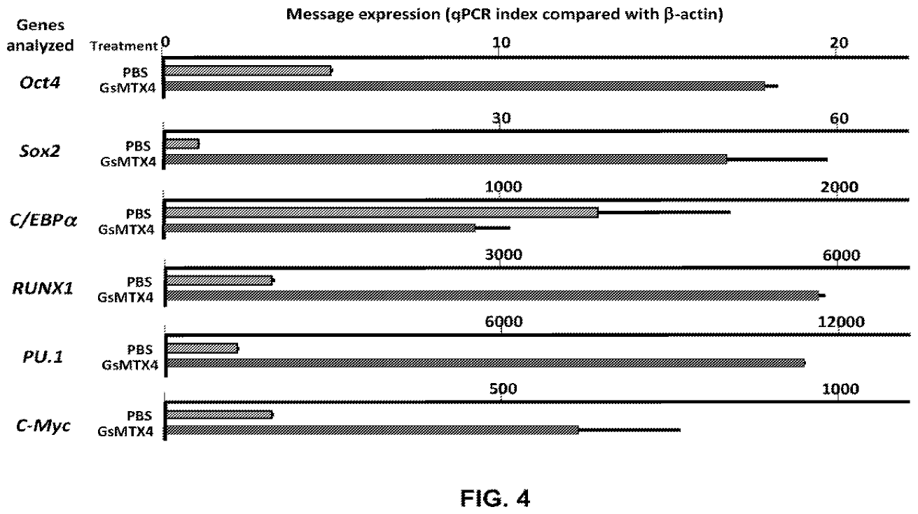

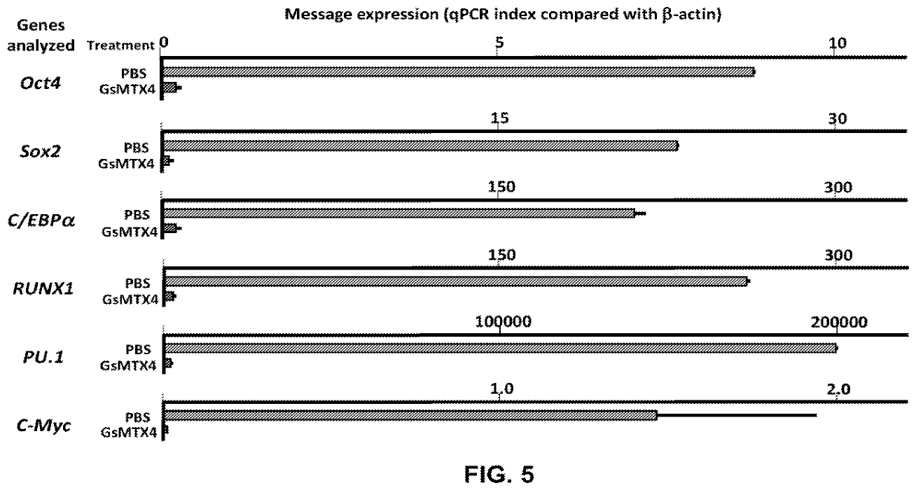

To investigate, more definitively, the cells possessing the opposite phenotypes when exposed to the same pressure and stretch stress, the study was extended by preparing a bone marrow-derived stromal cell line and an EF line. When bone marrow stroma cell line was O/N cultured in the presence of GsMTX4, the activation of PI genes in the cell resembled to that of fresh bone marrow cells (FIG. 4). Bone marrow stromal cell line were treated with GsMTX4 as described in FIG. 1. Gene expressions listed in the left were evaluated by the same methods as shown in FIG. 1. In the similar way, EF cells responded like that observed in the study of fresh spleen cells (FIG. 5). Embryonic fibroblast cells were treated with GsMTX4 as described in FIG. 1. Gene expressions listed in the left were evaluated by the same methods as shown in FIG. 1.

The data showed that the PI gene's regulation pattern induced by the mechanical stress could be inherited in the in vitro-adapted cell lines. The data also indicated that the specific phenotype in response to MSAIC signaling is not because of the transient mechanism resulting from the in vivo to in vitro adaptation but of an intrinsically programmed mechanism. This stable phenotype of the PI gene regulation by MSAICs may be relevant to the mechanism, which renders stem cells to retain the pluripotency.

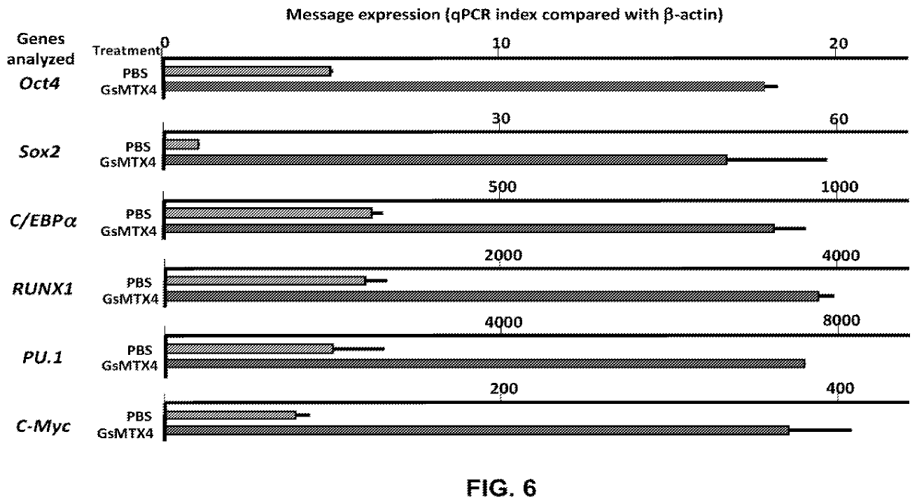

In further experiments the PI gene regulation in spleen cells and EF cells was investigated three days after the onset of the GsMTX4 treatment to obtain the insight if the repression is continuous. When assayed the PI gene expression the repression was no longer detected, instead, the activation of the PI genes was significantly observed (FIG. 6).

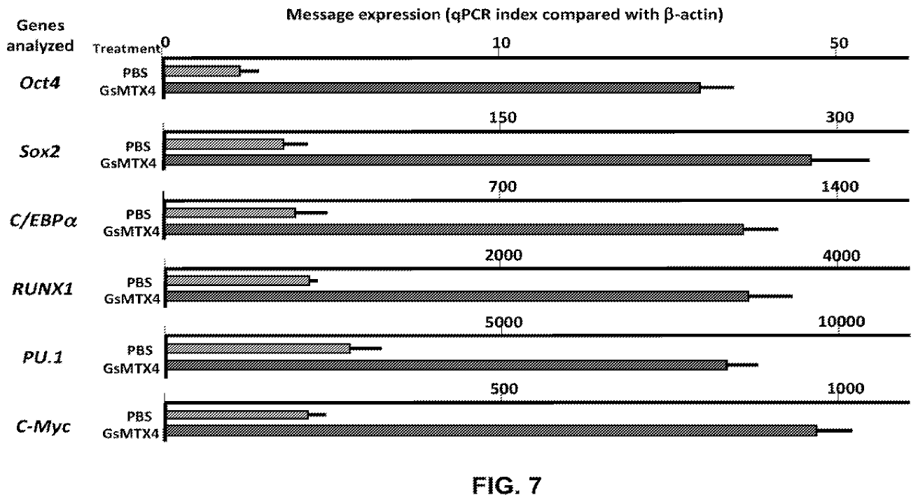

Spleen cells were cultured in 6 well plates as described in FIG. 1. Cells were harvested on day 3 and assayed for the expression of the pluripotent stem cell transcription factors listed in the left by the same methods as shown in FIG. 1. In the similar way, the expression of the PI genes in EF cells was found activated three days after the GsMTX4 treatment (FIG. 7). The EF cells were cultured in 6 well plates as described in FIG. 1. Cells were harvested on day 3 and assayed for the expression of the PIFs listed in the left by the same methods as shown in FIG. 1.

The data showed that the repression of PI genes in response to MSAIC inhibition was transient. Thus, the origin of the cells appeared to influence the early response against the mechanical stress measured by the PI genes' expression. The results seemed to reveal an unprecedented receptor mechanism, of which the same input of the stimulation causes entirely different outcome dependent on the type of cells, possibly because of the different stages of the cell differentiation.

The results indicated the presence of distinct type somatic cells following the attenuation of MSAIC signaling; one promptly activates the PI genes and the second responds in the delayed mode activate PI genes after the transient suppression. The data also showed that a prolonged attenuation of MSAIC signaling could reprogram both somatic cell types to acquire the stem cell like phenotypes possessing the high levels of PI genes.

Cholesterol depletion from spleen cell modified cells to respond against GsMTX4 in a resembling fashion to that of the bone marrow cells.

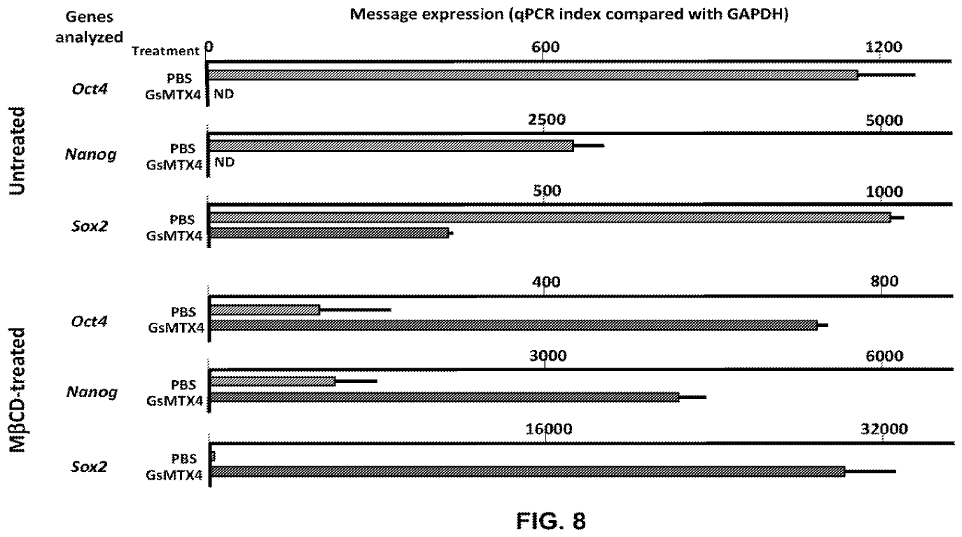

To obtain an insight into the molecular mechanism, which distinguishes spleen cells from bone marrow cells following the MSAIC inhibition, the roles of different stiffness and the signaling potential of cell membrane were investigated. In the physiological condition with a stable temperature, the cholesterol level, which is known to be higher in spleen than in bone marrow, would alter the cell membrane stiffness [Los and Murata, Biochim. Biophys. Acta. 1666:142 (2004); Simons and Sampaio, Cold Spring Harb. Perspect Biol. 3 (10) (2011)]. Cholesterol also plays a central role for the membrane receptor signaling by forming lipid rafts, in which MSAICs reside and initiate the mechanosensory signals [Szoke et al., Eur. J. Pharmacol. 628:67 (2010)]. Spleen cells were investigated for the expression of PI genes after the treatment with MβCD, which depletes cholesterol from cell membrane [Mahammad and Parmryd, Methods Mol. Biol. 1232: 91 (2015)]. Strikingly, the spleen cells treated with MβCD showed the activation of PI genes in response to GsMTX4 treatment in O/N culture (FIG. 8).

Spleen cells were 37° C.-treated with MβCD before the in vitro culture with GsMTX4 as described in FIG. 1. Gene expressions listed in the left were evaluated by the same methods as shown in FIG. 1. ND: not detected.

The results demonstrated that the level of the cholesterol in the cell membrane determined the resulting PI gene's expression in response to the MSAIC inhibition.

Conversion of EF cells to pluripotent stem cell-like cells by the depletion of cellular lipids and/or soft extra-cellular matrix.

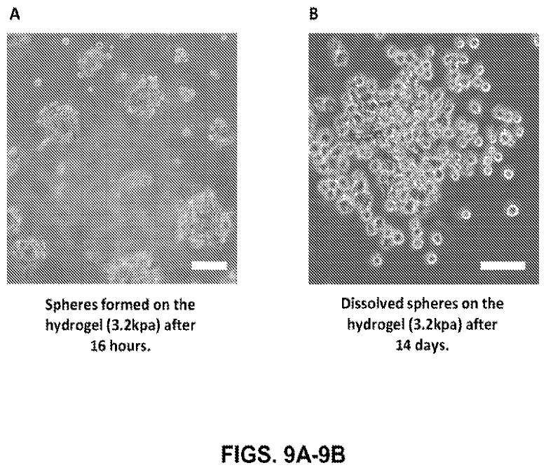

A cell culture method was developed by which cells acquired pluripotent stem cell-activity, as was seen with EF cells gene-transfected with PI genes [Takahashi and Yamanaka, Cell, 126:663 (2006)]. Cells were treated with MβCD, then seeded on the low pascal hydrogels (1,7 kpa, 3.2 kpa and 7.4 kpa). Within 60 min, after the initiation of the culture, cells adhered tightly to the hydrogel, then the formation of various size spheres were observed in the O/N culture (FIG. 9A).

To investigate the expression of PI genes, the total RNAs from the sphere and the well bottom-adhered cells (spilled from the gel surface during the seeding procedure) were RT-PCR characterized by 1.5% agarose gel electrophoresis.

EF cells were MβCD-treated and cultured in the hydrogel-casted petri dishes for 7 days before RNA extraction. The stiffness of the hydrogel was listed on the top of each lane. Total RNAs from spheres and dish-adhered cells were analyzed for the expression of the PI genes by RT-PCR. PCR-amplified fragments were dissolved by 1.5% agarose gel and stained by Ethidium Bromide. Mouse embryonic stem (mES) cell total RNA was used as the positive control. From left to right, a; Oct4 analysis, b; Nanog analysis, c; Sox2 analysis.

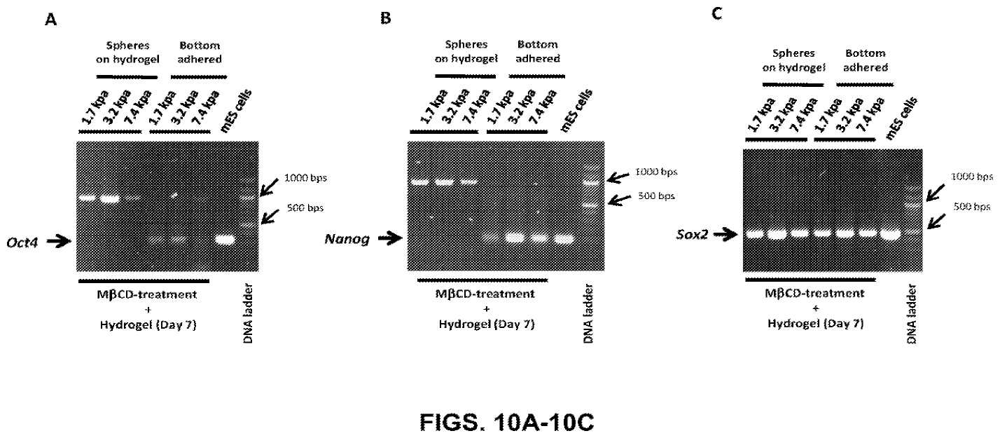

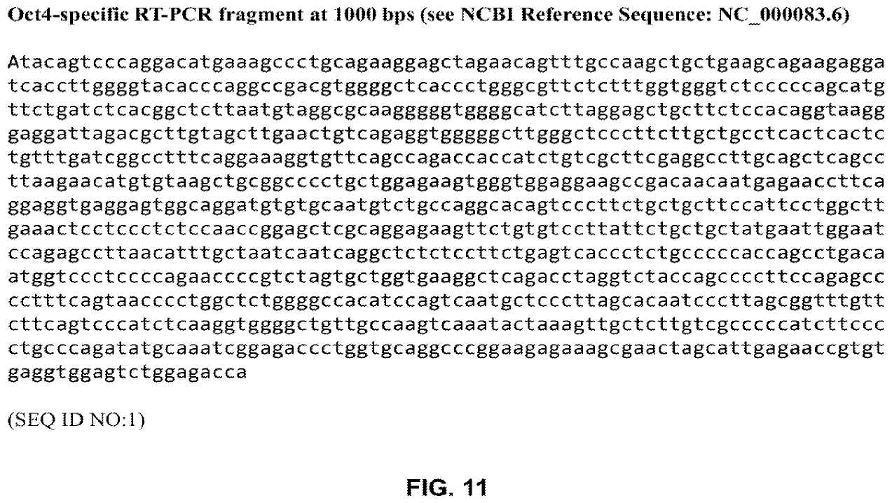

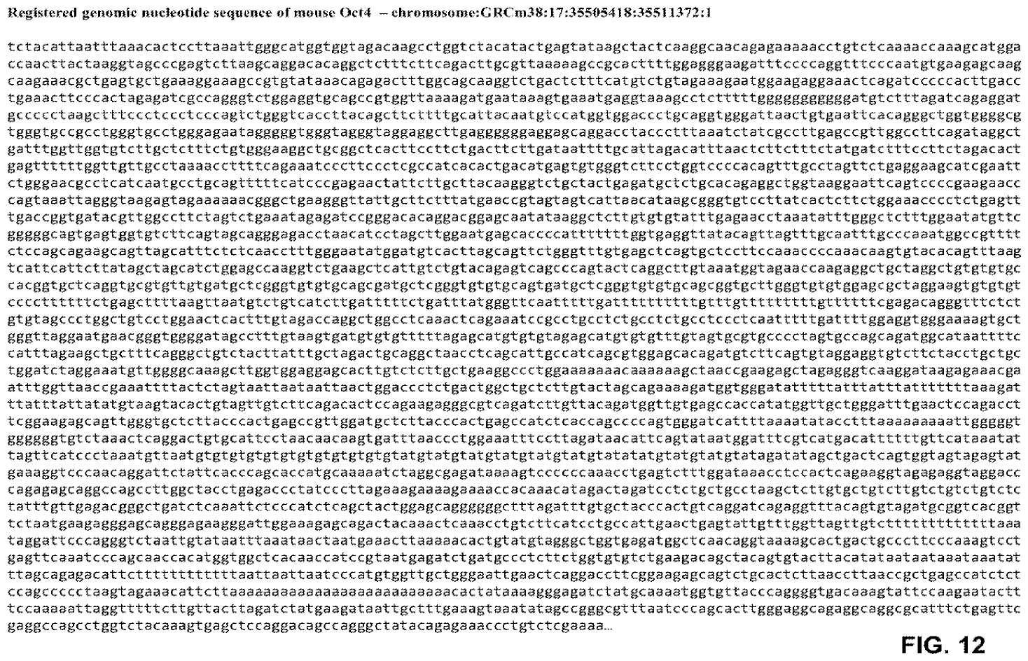

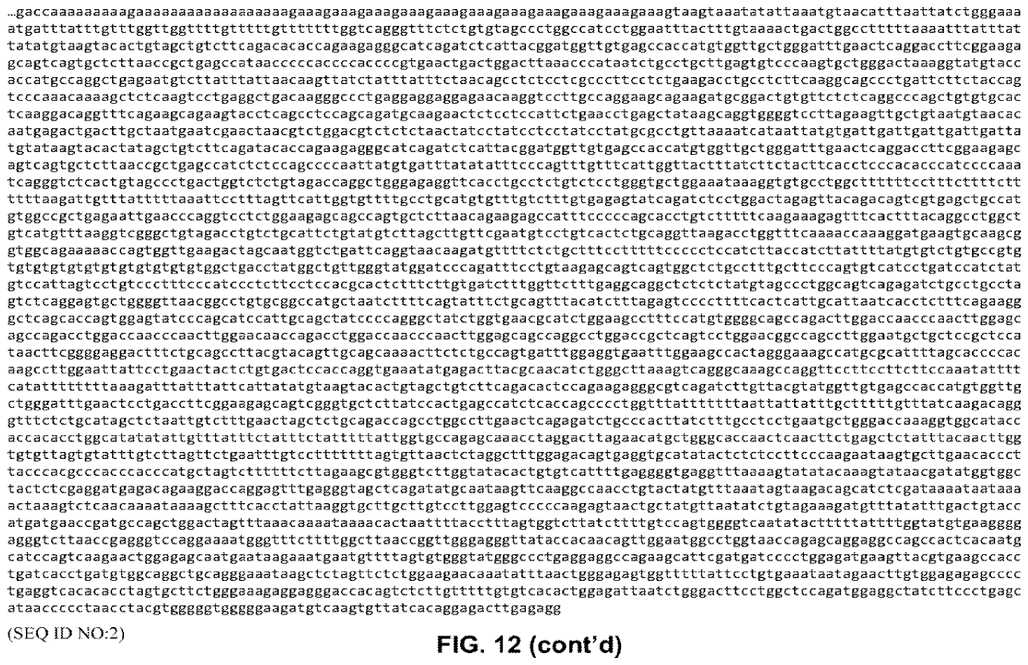

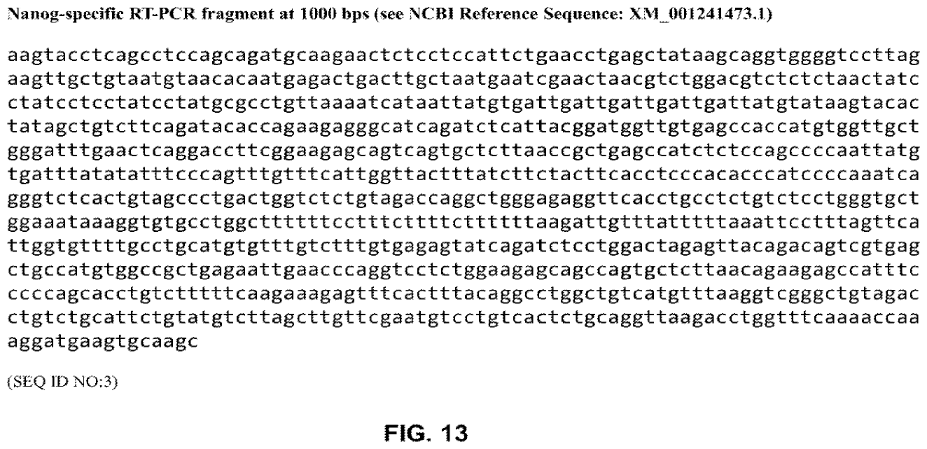

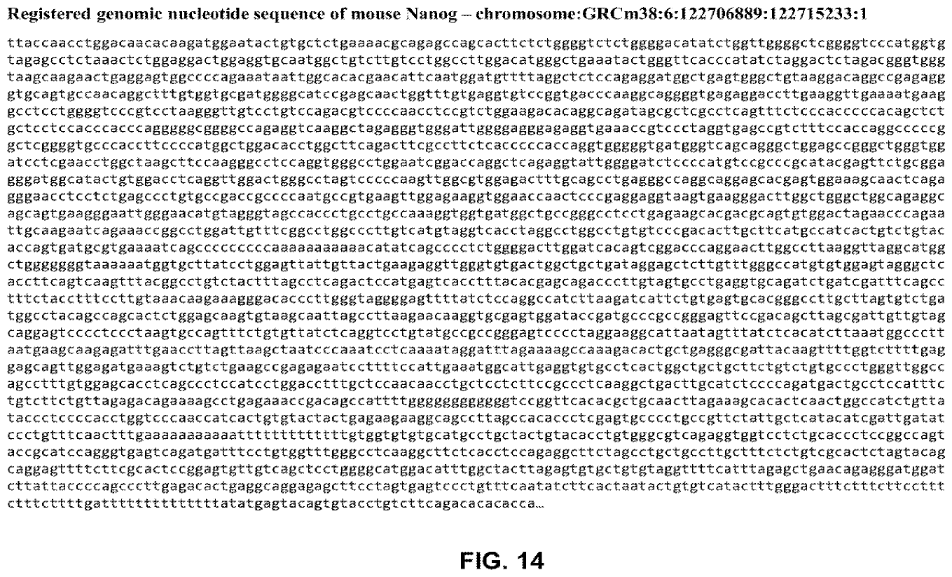

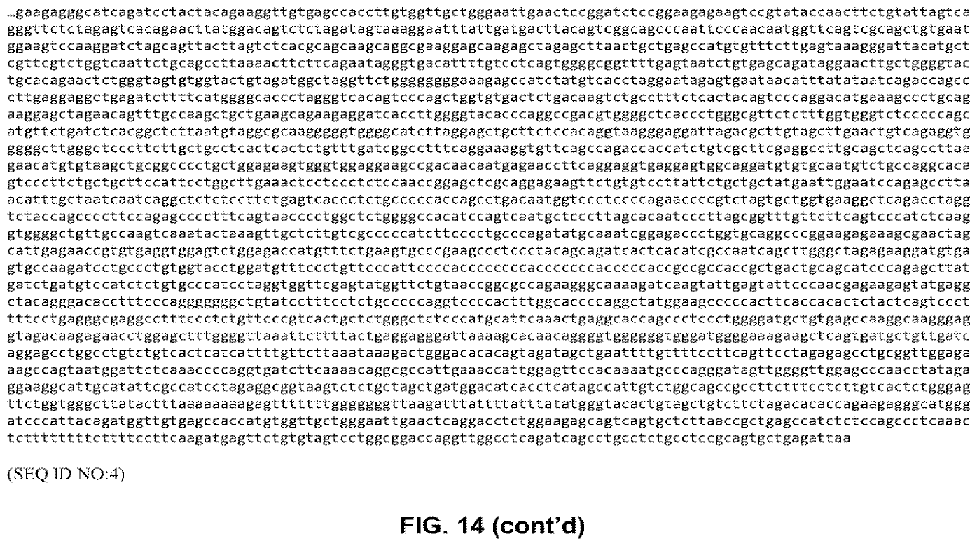

As shown in the FIG. 10, a week-old culture showed significant messages for Oct4, Nanog, and Sox2 in the bottom-adhered cells at the predicted sizes but the RNAs obtained from the spheres did not show the predicted bands specific for Oct4, instead, showed prominent bands at 1000 bps (FIG. 10A). These bands, however, completely disappeared when the predicted size bands appeared. Similar results were also observed in the assays for Nanog expression (FIG. 10B). However, Sox2, which is an intronless gene did not show large size bands [Nagai, Jpn. J. Hum. Genet. 41:363 (1996)] (FIG. 10C). It was hypothesized that those 1000 bps bands in Oct4 and Nanog-specific RT-PCR studies represented the fragments originated from the non-spliced messages. The results from the sequencing of the purified 1000 bps fragments (FIG. 11) (SEQ ID NO:1), indeed, matched to the reported sequences, which encompass the exon 3 and exon 5 of Oct4 (FIG. 12) (SEQ ID NO:2) and exon 2 and exon 3 of Nanog (FIG. 13) (SEQ ID NO:3) including the introns (FIG. 14) (SEQ ID NO:4). Correspondingly, the presence of the un-spliced Oct4 precursor mRNA of the mouse in the cancer cells was designated as Oct4a in the report previously [Liu et al., J. Cell. Physiol. 233:5468 (2018)].

The study was extended by investigating the activation kinetics of the PIF messages induced in the cells attached to the well bottom surface (FIGS. 15A-15B).

EF cells were MβCD-treated and cultured in the petri dishes. Microscopic morphological views at day 1, day 4 and day 7 after the onset of the experiment was shown in A. The total RNA was extracted at the time points listed on the top. The message for each PI gene was analyzed as described in FIG. 1 and shown in B.

Surprisingly, un-spliced messages within 24 hours after the cholesterol depletion were detected. The spliced messages were present only in the cells cultured 7 days. The results indicated that 1) the cholesterol depletion activated PI genes precursor transcription within 24 hours, 2) the expression of the spliced messages starts later after 4 days but before 7 days and 3) the soft hydrogel prevented the splicing of the precursor messages of the PI genes. Thus, the softness of the extracellular matrix profoundly controlled the activation and splicing mechanisms of PI genes in somatic cells.

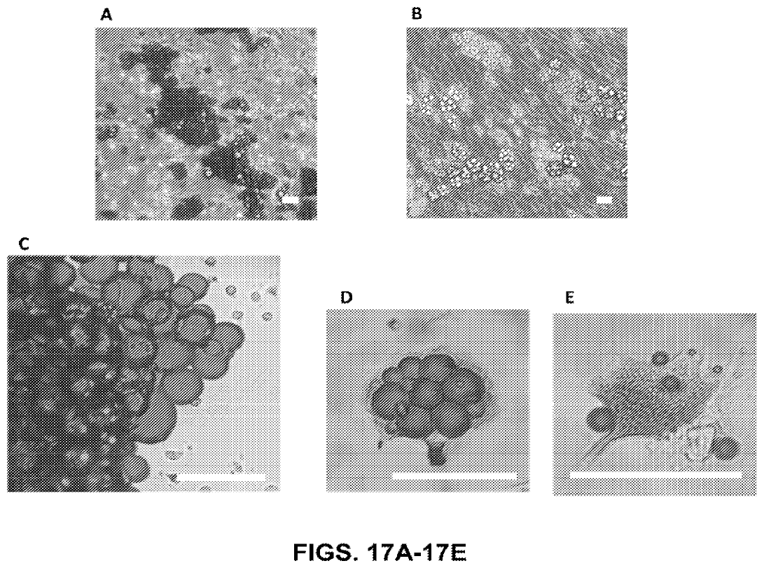

To investigate if the differentiation of new phenotype cells follows in the continuing culture from those PI gene-activated pluripotent stem cell-like cells, cells were cultured for an extended period. Within fourteen days, most of the spheres on the gel dissolved to the clusters of round cells (FIG. 9B). Concomitantly, the adhesion of cells to the hydrogel appeared to become weak and cells were released to the dish bottom. Strikingly, the differentiation of unique-phenotype cells was observed on the bottom of the culture dish within a week. We hardly observed the proliferation or morphological change of hydrogel attached cells in the EF cell studies. Thus, the results suggested that the cells seeded on the petri dish surface and those spilled from the acrylamide gel to the petri dish surface differentiated into the unique-phenotype cells. The examples of the unique-phenotype cells appeared on the dish bottom were shown in FIG. 17 to FIG. 27.

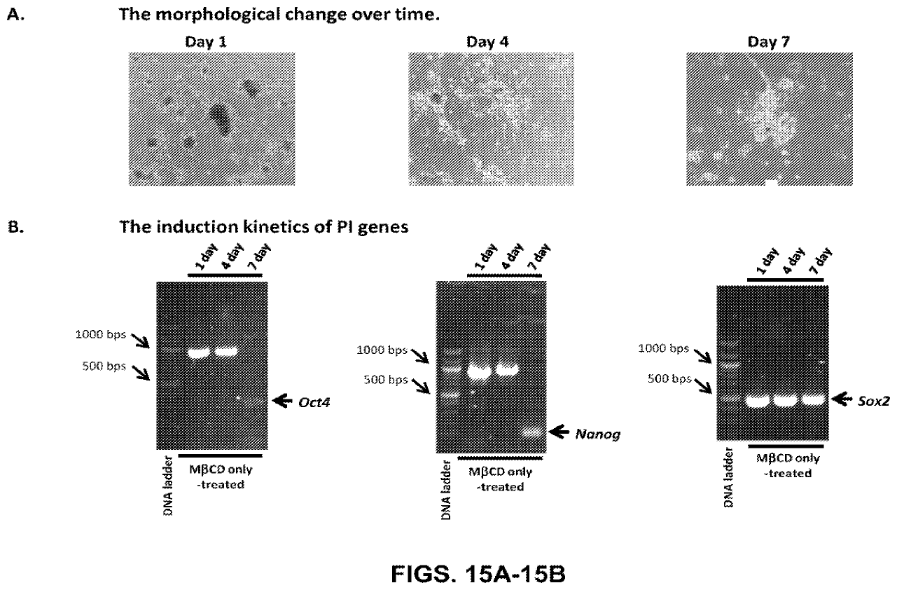

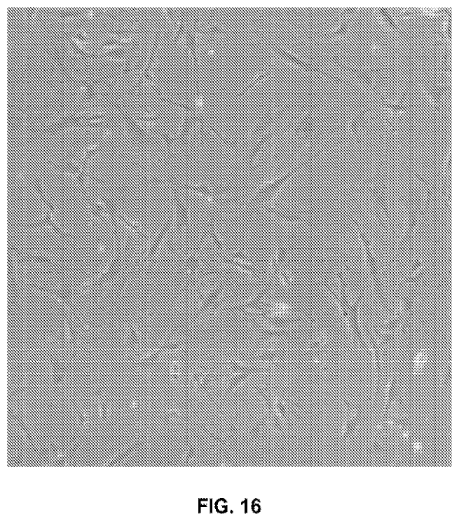

Specifically, the EF cells were MβCD-treated and cultured in 3,2 kpa hydrogel-casted petri dishes. Cells differentiated on the dish bottom were photographed as they were observed. Based on the reported phenotypes of the different lineage cells, each cell unique from the original EF cell (FIG. 16) was tentatively labeled as shown above the pictures. The cells obviously different from the original EF cells but were unable to assign the specific cell type name were labeled as “Not classified”. White bars in panels indicate 100 μm size references.

Clusters of the cells resembled immature adipocytes, and those cells transformed to the mature adipocytes within several days (FIGS. 17A-17B). Those cells were positively stained with the lipid-specific dye, Oil red 0 (FIG. 17C-17E).

Brown adipocytes (FIG. 17A) and white adipocytes (FIG. 17B) were frequently observed through the culture after 10 days. To characterize them as adipocytes, cells were stained with Oil red 0 (FIG. 17C-E).

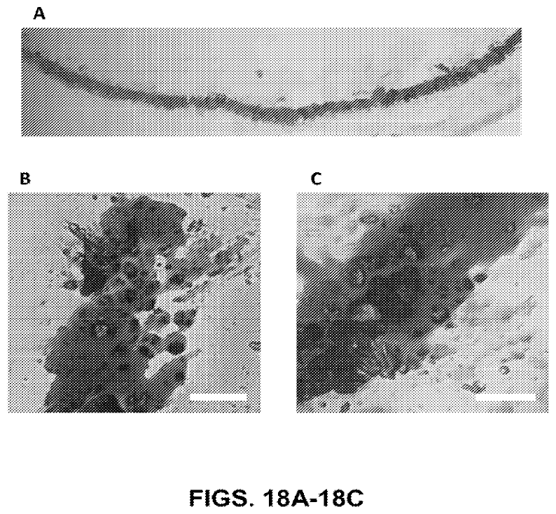

On the other hand, the staining with Alizalin demonstrated the calcium deposits and osteoblastic cells in the specific area of the culture (FIGS. 18A-18C).

The presence of the osteoblasts/osteocytes was demonstrated with Alizalin staining and shown in FIGS. 18A-188C.

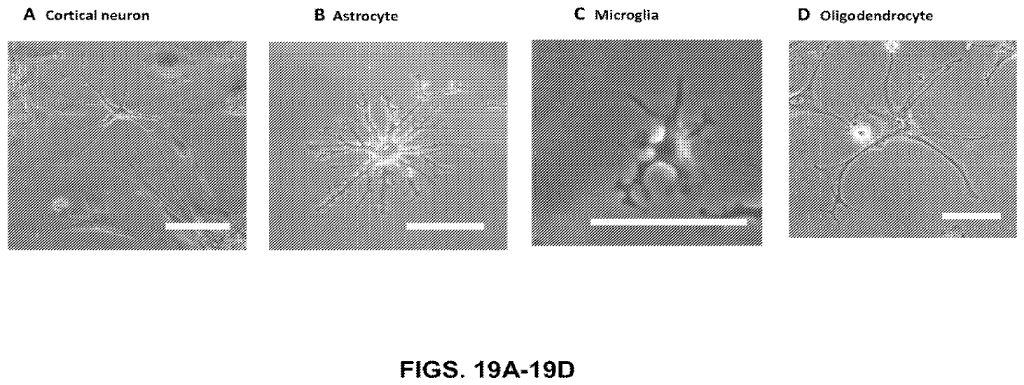

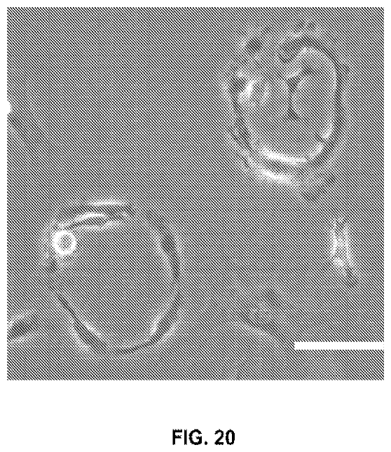

Neuronal cell-like cells at the size 100-500 μm (FIGS. 19A-19D) and endothelial cell-like cells (FIG. 20) were observed.

Often observed within 10 days were the neuronal cells including cortical neuron-like cells (FIG. 19A), astrocyte-like cells (FIG. 19B), microglia-like cells (FIG. 19C) and oligodendrocyte-like cells (FIG. 19D). Circle-connected endothelial cell-like cells were also observed in the same timing (FIG. 20).

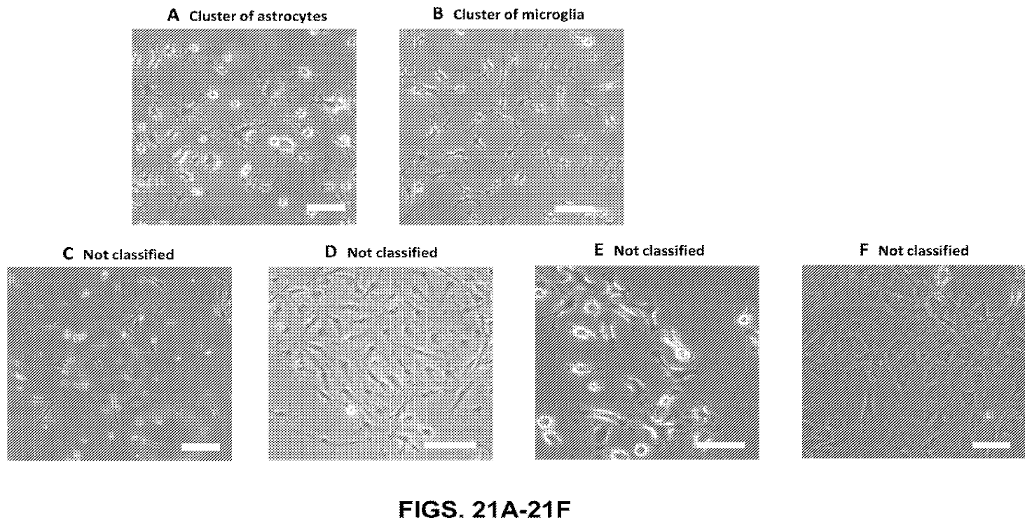

Similar phenotype cells tended to cluster in the specific areas of the bottom surface (FIG. 21A-21F).

FIG. 21A-2F shows colonies of similar phenotype cells differentiated from mouse EF cells treated with MβCD and hydrogel.

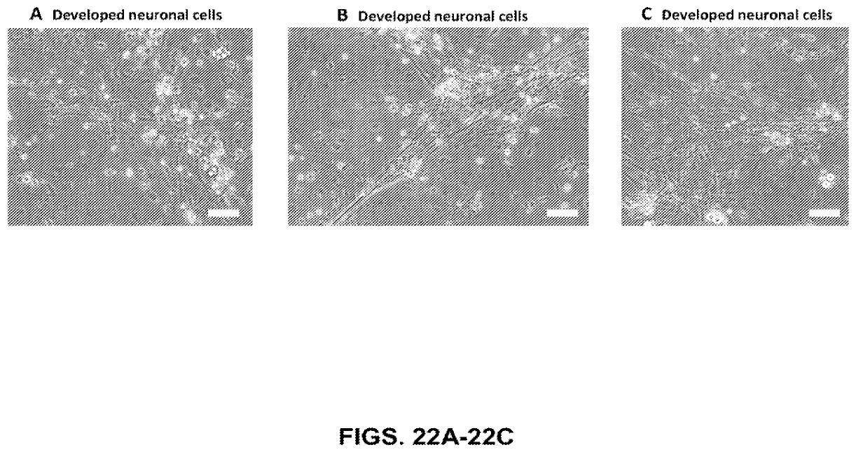

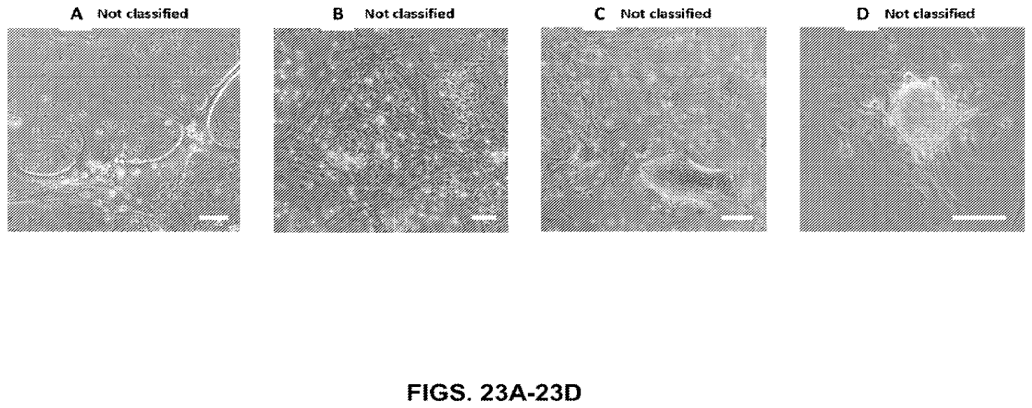

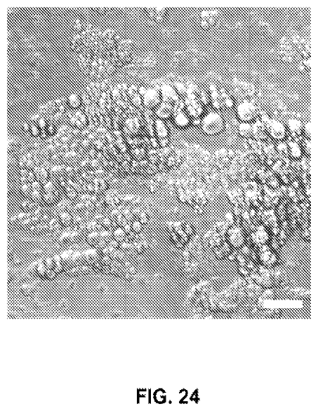

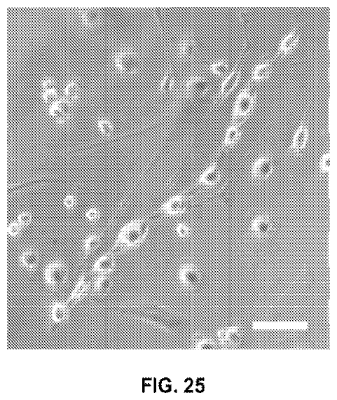

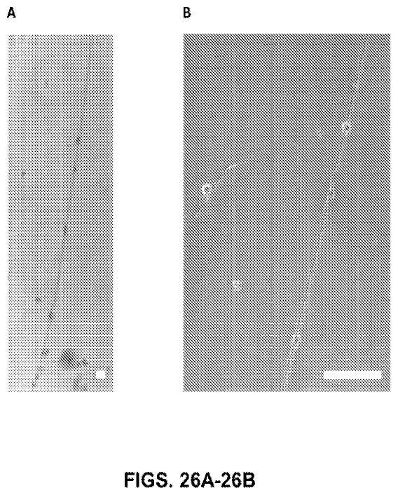

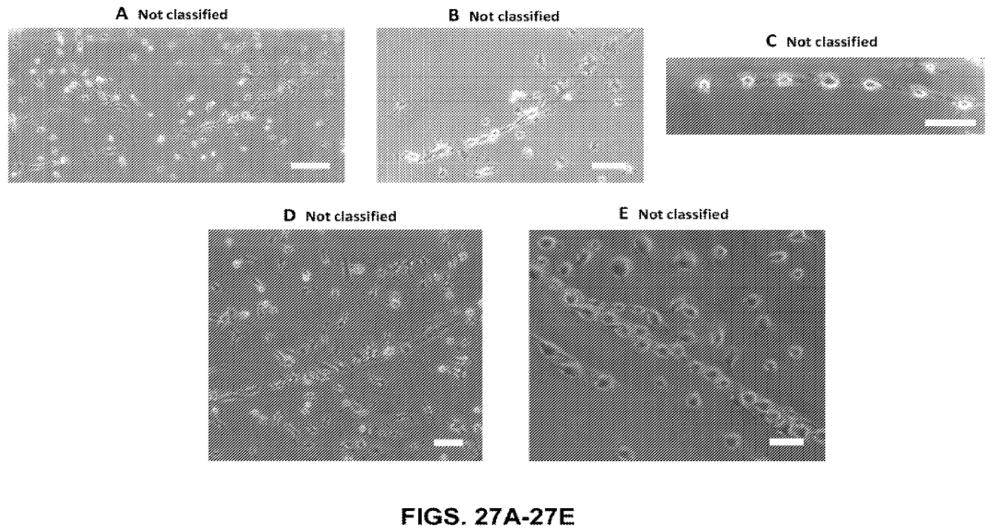

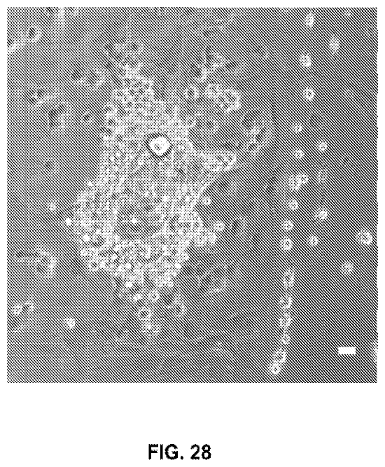

Later after 3 weeks, the well-developed neuronal cell clusters (FIGS. 22A-22C) and uniquely aggregated non-classified cell clusters became remarkable (FIG. 23). Well-matured adipocyte masses became evident as well (FIG. 24). Later after 4 weeks, myoblast-like cells (FIG. 25), and tendon cell/progenitor-like cells became noticeable (FIGS. 26A-26B). We also observed chains of the cells interacted to build microstructures after 4 weeks of the culture (FIG. 27A-27E).

Five examples of the cells interacted and formed the alignment were shown.

Thus, the results suggested that the pluripotent stem cell-like cells derived from EF cells vigorously differentiated into various lineage cells in vitro.

Spheres were harvested from the surface of 3.2 kpa acrylamide gel with Trypsin treatment as described in FIG. 9. Cells were washed and re-seeded on the polystyrene surface in the 24 well tissue culture plate.

Hydrogel alone can activate PI genes in EFs to generate stem cell-like cells, but the efficiency is low and takes long incubation time before reprogrammed and differentiated cells start populating on the bottom surface of the petri dish.

MβCD only-treatment induces stem cell-like cells within a week with a greater efficiency than that of the hydrogel only.

Combined treatment with MβCD and the hydrogel results in the highest efficiency to the development of differentiated cells on the bottom of petri dish.

The Examples presented supra indicated that the current invention can reprogram somatic cells to pluripotent stem cell-like cells, which possess functions to differentiate to various phenotype cells with different functions. The discovery is that MSAIC signal repress the PI gene expression and the inhibition of the MSAIC signal render somatic cells express PI genes and acquire the pluripotency. The discovery taught multiple methods to attenuate MSAIC signaling in somatic cells: specific inhibitor, GsMTX4, soft polyacrylamide gel and the cholesterol depletion with MβCD. All methods are capable to activate PI genes and reprogram somatic cells. The discovery also taught that the combined use of different methods potentiates the reprogramming of somatic cells. The discovery taught the re-differentiation of the pluripotent stem cell-like cell generates large repertoire of somatic cells. All those differentiated cells are expected to be useful for the regenerative therapy. Among them, for example, osteocytes are useful to reconstitute bone fracture and neuronal cells are expected to restore the neuronal injury and the brain diseases, Parkinson disease and Alzheimer's diseases. It is also anticipated that, based on these examples, the present invention will provide novel methods of treatment of diseases that either enhance or repress cellular regeneration. The invention will broadly encompass the use of the PS-like cells and the re-differentiated cells for treatment or prevention of diseases wherein enhanced presence of specific type somatic cell is desirable.

It is to be understood that the invention is not limited to the embodiments listed above and the right is reserved to the illustrated embodiments and all modifications coming within the scope of the following claims.

The various references to journals, patents, and other publications which are cited herein comprise the state of the art and are incorporated by reference as though fully set forth.

1. A method of inducing a non-pluripotent mammalian cell into an induced pluripotent stem cell, the method comprising contacting the non-pluripotent mammalian cell with two or more of the following:
a. one or more mechanosensitive and stretch-activated ion channel inhibitors in an amount sufficient to inhibit the mammalian cell ion channels;
b. one or more cell cholesterol reducing agents in an amount sufficient to reduce the mammalian cell cholesterol level;
c. a soft extracellular matrix having a Young's elastic modulus of 20 kPa or less.

2. The method of claim 1, wherein the non-pluripotent mammalian cell is not genetically modified to express pluripotency inducing factors.

3. The method of claim 1, wherein the mechanosensitive and stretch-activated ion channel inhibitor is selected from the group consisting of the L enantiomer of GsMTX4, the D enantiomer of GsMTX4, a peptide having a sequence at least 90% identical to the sequence of GsMTX4, or a mixture thereof.

4. The method of claim 1, wherein the mechanosensitive and stretch-activated ion channel inhibitor is GsMTX4.

5. The method of claim 3, wherein the mechanosensitive and stretch-activated ion channel inhibitor is at a concentration of about 5 μM.

6. The method of claim 1, wherein the cell cholesterol reducing agent is a cyclodextrin.

7. The method of claim 6, wherein the cyclodextrin is methyl-β-cyclodextrin.

8. The method of claim 7, wherein the cyclodextrin is at a concentration of about 5 mM.

9. The method of claim 1, wherein the extracellular matrix has a Young's elastic modulus of about 15 kPa or less.

10. The method of claim 1, wherein the extracellular matrix has a Young's elastic modulus of about 7.4 kPa or less.

11. The method of claim 1, wherein the induced pluripotent stem cell is capable of differentiating into a cell type selected from the group consisting of adipocytes, neuronal cells, osteocytes, endothelial cells, erythrocytes, dendritic cells, platelets, lymphocytes, and myoblasts.

12. The method of claim 1, wherein the expression of one or more of the genes Oct4, Nanog and Sox2 is induced in the induced pluripotent stem cell relative to the non-pluripotent mammalian cell.

13. A pharmaceutical composition comprising an isolated population of cells having a second non-pluripotent cell type, wherein the cells are obtained by a composition of converting animal cells from a first non-pluripotent cell type, and wherein the composition comprises inducing a non-pluripotent mammalian cell of a first cell type into an induced pluripotent stem cell by
a. contacting the non-pluripotent mammalian cell with two or more of the following:
i. one or more mechanosensitive and stretch-activated ion channel inhibitors in an amount sufficient to inhibit the mammalian cell ion channels;
ii. one or more cell cholesterol reducing agents in an amount sufficient to reduce the mammalian cell cholesterol level;
iii. a soft extracellular matrix having a Young's elastic modulus of 20 kPa or less, and
b. inducing differentiation of the cells from step (a) into the second non-pluripotent cell type.

14. A cell culture container comprising
a. cell culture media,
b. one or more mammalian cells treated with one or both of the following:
i. one or more mechanosensitive and stretch-activated ion channel inhibitors in an amount sufficient to inhibit the mammalian cell ion channels;
ii. one or more cell cholesterol reducing agents in an amount sufficient to reduce the mammalian cell cholesterol level; and
c. a soft extracellular matrix having a Young's elastic modulus of 20 kPa or less.


SEQUENCE LISTING (28 sequences)

SEQ ID NO: 1 - DNA, 969 bp, Artificial Sequence
  Synthetic polynucleotide
ctacagtcccaggacatgaaagccctgcagaaggagctagaacagtttgccaagctgctgaagcagaagaggatcaccttggggtacacccaggccgacgtggggctcaccctgggcgttctctttggtgggtctcccccagcatgttctgatctcacggctcttaatgtaggcgcaagggggtggggcatcttaggagctgcttctccacaggtaagggaggattagacgcttgtagcttgaactgtcagaggtgggggcttgggctcccttcttgctgcctcactcactctgtttgatcggcctttcaggaaaggtgttcagccagaccaccatctgtcgcttcgaggccttgcagctcagccttaagaacatgtgtaagctgcggcccctgctggagaagtgggtggaggaagccgacaacaatgagaaccttcaggaggtgaggagtggcaggatgtgtgcaatgtctgccaggcacagtcccttctgctgcttccattcctggcttgaaactcctccctctccaaccggagctcgcaggagaagttctgtgtccttattctgctgctatgaattggaatccagagccttaacatttgctaatcaatcaggctctctccttctgagtcaccctctgcccccaccagcctgacaatggtccctccccagaaccccgtctagtgctggtgaaggctcagacctaggtctaccagccccttccagagcccctttcagtaacccctggctctggggccacatccagtcaatgctcccttagcacaatcccttagcggtttgttcttcagtcccatctcaaggtggggctgttgccaagtcaaatactaaagttgctcttgtcgcccccatcttcccctgcccagatatgcaaatcggagaccctggtgcaggcccggaagagaaagcgaactagcattgagaaccgtgtgaggtggagtctggagacca

SEQ ID NO: 2 - DNA, 8345 bp, Mus musculus
tctacattaatttaaacactccttaaattgggcatggtggtagacaagcctggtctacatactgagtataagctactcaaggcaacagagaaaaacctgtctcaaaaccaaagcatggaccaacttactaaggtagcccgagtcttaagcaggacacaggctctttcttcagacttgcgttaaaaagccgcacttttggagggaagatttccccaggtttcccaatgtgaagagcaagcaagaaacgctgagtgctgaaaggaaagccgtgtataaacagagactttggcagcaaggtctgactctttcatgtctgtagaaagaatggaagaggaaactcagatcccccacttgacctgaaacttcccactagagatcgccagggtctggaggtgcagccgtggttaaaagatgaataaagtgaaatgaggtaaagcctctttttgggggggggggatgtctttagatcagaggatgccccctaagctttccctccctcccagtctgggtcaccttacagcttcttttgcattacaatgtccatggtggaccctgcaggtgggattaactgtgaattcacagggctggtggggcgtgggtgccgcctgggtgcctgggagaatagggggtgggtagggtaggaggcttgaggggggaggagcaggacctaccctttaaatctatcgccttgagccgttggccttcagataggctgatttggttggtgtcttgctctttctgtgggaaggctgcggctcacttccttctgacttcttgataattttgcattagacatttaactcttctttctatgatctttccttctagacactgagttttttggttgttgcctaaaaccttttcagaaatcccttccctcgccatcacactgacatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctggtaaggaattcagtccccgaagaacccagtaaattagggtaagagtagaaaaaacgggctgaagggttattgcttctttatgaaccgtagtagtcattaacataagcgggtgtccttatcactcttctggaaacccctctgagtttgaccggtgatacgttggccttctagtctgaaatagagatccgggacacaggacggagcaatataaggctcttgtgtgtatttgagaacctaaatatttgggctctttggaatatgttcgggggcagtgagtggtgtcttcagtagcagggagacctaacatcctagcttggaatgagcaccccatttttttggtgaggttatacagttagtttgcaatttgcccaaatggccgttttctccagcagaagcagttagcatttctctcaacctttgggaatatggatgtcacttagcagttctgggtttgtgagctcagtgctccttccaaaccccaaacaagtgtacacagtttaagtcattcattcttatagctagcatctggagccaaggtctgaagctcattgtctgtacagagtcagcccagtactcaggcttgtaaatggtagaaccaagaggctgctaggctgtgtgtgccacggtgctcaggtgcgtgttgtgatgctcgggtgtgtgcagcgatgctcgggtgtgtgcagtgatgctcgggtgtgtgcagcggtgcttgggtgtgtggagcgctaggaagtgtgtgtccccttttttctgagcttttaagttaatgtctgtcatcttgatttttctgatttatgggttcaatttttgattttttttttgtttgtttttttttgttttttcgagacagggtttctctgtgtagccctggctgtcctggaactcactttgtagaccaggctggcctcaaactcagaaatccgcctgcctctgcctctgcctccctcaatttttgattttggaggtgggaaaagtgctgggttaggaatgaacgggtggggatagcctttgtaagtgatgtgtgtttttagagcatgtgtgtagagcatgtgtgtttgtagtgcgtgcccctagtgccagcagatggcataattttccatttagaagctgctttcagggctgtctacttatttgctagactgcaggctaacctcagcattgccatcagcgtggagcacagatgtcttcagtgtaggaggtgtcttctacctgctgctggatctaggaaatgttggggcaaagcttggtggaggagcacttgtctcttgctgaaggccctggaaaaaaacaaaaaagctaaccgaagagctagagggtcaaggataagagaaacgaatttggttaaccgaaattttactctagtaattaataattaactggaccctctgactggctgctcttgtactagcagaaaagatggtgggatatttttatttatttatttttttaaagatttatttattatatgtaagtacactgtagttgtcttcagacactccagaagagggcgtcagatcttgttacagatggttgtgagccaccatatggttgctgggatttgaactccagaccttcggaagagcagttgggtgctcttacccactgagccgttggatgctcttacccactgagccatctcaccagccccagtgggatcattttaaaatatacctttaaaaaaaaattgggggtggggggtgtctaaactcaggactgtgcattcctaacaacaagtgatttaaccctggaaatttccttagataacattcagtataatggatttcgtcatgacattttttgttcataaatattagttcatccctaaatgttaatgtgtgtgtgtgtgtgtgtgtgtgtatgtatgtatgtatgtatgtatgtatatatgtatgtatgtatagatatagctgactcagtggtagtagagtatgaaaggtcccaacaggattctattcacccagcaccatgcaaaaatctaggcgagataaaagtccccccaaacctgagtctttggataaacctccactcagaaggtagagaggtaggacccagagagcaggccagccttggctacctgagaccctatcccttagaaagaaaagaaaaccacaaacatagactagatcctctgctgcctaagctcttgtgctgtcttgtctgtctgtctctatttgttgagacgggctgatctcaaattctcccatctcagctactggagcaggggggctttagatttgtgctacccactgtcaggatcagaggtttacagtgtagatgcggtcacggttctaatgaagagggagcagggagaagggattggaaagagcagactacaaactcaaacctgtcttcatcctgccattgaactgagtattgtttggttagttgtctttttttttttttaaataggattcccagggtctaattgtataatttaaataactaatgaaacttaaaaacactgtatgtagggctggtgagatggctcaacaggtaaaagcactgactgcccttcccaaagtcctgagttcaaatcccagcaaccacatggtggctcacaaccatccgtaatgagatctgatgccctcttctggtgtgtctgaagacagctacagtgtacttacatataataataaataaatatttagcagagacattcttttttttttttaattaattaatcccatgtggttgctgggaattgaactcaggaccttcggaagagcagtctgcactcttaaccttaaccgctgagccatctctccagccccctaagtagaaacattcttaaaaaaaaaaaaaaaaaaaaaaaaaacactataaaagggagatctatgcaaaatggtgttacccaggggtgacaaagtattccaagaatactttccaaaaattaggtttttcttgttacttagatctatgaagataattgctttgaaagtaaatatagccgggcgtttaatcccagcacttgggaggcagaggcaggcgcatttctgagttcgaggccagcctggtctacaaagtgagctccaggacagccagggctatacagagaaaccctgtctcgaaaaaaccaaaaaaaaagaaaaaaaaaaaaaaaaaagaaagaaagaaagaaagaaagaaagaaagaaagaaagaaagaaagtaagtaaatatattaaatgtaacatttaattatctgggaaaatgatttatttgtttggttggttttgtttttgtttttttggtcagggtttctctgtgtagccctggccatcctggaatttactttgtaaaactgactggcctttttaaaatttatttattatatgtaagtacactgtagctgtcttcagacacaccagaagagggcatcagatctcattacggatggttgtgagccaccatgtggttgctgggatttgaactcaggaccttcggaagagcagtcagtgctcttaaccgctgagccataacccccaccccaccccgtgaactgactggacttaaacccataatctgcctgcttgagtgtcccaagtgctgggactaaaggtatgtaccaccatgccaggctgagaatgtcttatttattaacaagttatctatttatttctaacagcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggtggggtccttagaagttgctgtaatgtaacacaatgagactgacttgctaatgaatcgaactaacgtctggacgtctctctaactatcctatcctcctatcctatgcgcctgttaaaatcataattatgtgattgattgattgattgattatgtataagtacactatagctgtcttcagatacaccagaagagggcatcagatctcattacggatggttgtgagccaccatgtggttgctgggatttgaactcaggaccttcggaagagcagtcagtgctcttaaccgctgagccatctctccagccccaattatgtgatttatatatttcccagtttgtttcattggttactttatcttctacttcacctcccacacccatccccaaatcagggtctcactgtagccctgactggtctctgtagaccaggctgggagaggttcacctgcctctgtctcctgggtgctggaaataaaggtgtgcctggcttttttcctttcttttcttttttaagattgtttatttttaaattcctttagttcattggtgttttgcctgcatgtgtttgtctttgtgagagtatcagatctcctggactagagttacagacagtcgtgagctgccatgtggccgctgagaattgaacccaggtcctctggaagagcagccagtgctcttaacagaagagccatttcccccagcacctgtctttttcaagaaagagtttcactttacaggcctggctgtcatgtttaaggtcgggctgtagacctgtctgcattctgtatgtcttagcttgttcgaatgtcctgtcactctgcaggttaagacctggtttcaaaaccaaaggatgaagtgcaagcggtggcagaaaaaccagtggttgaagactagcaatggtctgattcaggtaacaagatgttttctctgctttcctttttccccctccatcttaccatcttattttatgtgtctgtgccgtgtgtgtgtgtgtgtgtgtgtgtgtggctgacctatggctgttgggtatggatcccagatttcctgtaagagcagtcagtggctctgcctttgcttcccagtgtcatcctgatccatctatgtccattagtcctgtccctttcccatccctcttcctccacgcactctttcttgtgatctttggttctttgaggcaggctctctctatgtagccctggcagtcagagatctgcctgcctagtctcaggagtgctggggttaacggcctgtgcggccatgctaatcttttcagtatttctgcagtttacatctttagagtccccttttcactcattgcattaatcacctctttcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaagccaggttccttccttcttccaaatattttcatattttttttaaagatttatttattcattatatgtaagtacactgtagctgtcttcagacactccagaagagggcgtcagatcttgttacgtatggttgtgagccaccatgtggttgctgggatttgaactcctgaccttcggaagagcagtcgggtgctcttatccactgagccatctcaccagcccctggtttatttttttaattattatttgctttttgtttatcaagacagggtttctctgcatagctctaattgtctttgaactagctctgcagaccagcctggccttgaactcagagatctgcccacttatctttgcctcctgaatgctgggaccaaaggtggcataccaccacacctggcatatatattgtttatttctatttctatttttattggtgccagagcaaacctaggacttagaacatgctgggcaccaactcaacttctgagctctatttacaacttggtgtgttagtgtatttgtcttagttctgaatttgtcctttttttagtgttaactctaggctttggagacagtgaggtgcatatactctctccttcccaagaataagtgcttgaacacccttacccacgcccacccacccatgctagtcttttttcttagaagcgtgggtcttggtatacactgtgtcattttgaggggtgaggtttaaaagtatatacaaagtataacgatatggtggctactctcgaggatgagacagaaggaccaggagtttgagggtagctcagatatgcaataagttcaaggccaacctgtactatgtttaaatagtaagacagcatctcgataaaataataaaactaaagtctcaacaaaataaaagctttcacctattaaggtgcttgcttgtccttggagtcccccaagagtaactgctatgttaatatctgtagaaagatgtttatatttgactgtaccatgatgaaccgatgccagctggactagtttaaacaaaataaaacactaattttacctttagtggtcttatcttttgtccagtggggtcaatatactttttattttggtatgtgaaggggagggtcttaaccgagggtccaggaaaatgggtttcttttggcttaaccggttgggagggttataccacaacagttggaatggcctggtaaccagagcaggaggccagccactcacaatgcatccagtcaagaactggagagcaatgaataagaaatgaatgttttagtgtgggtatgggccctgaggaggccagaagcattcgatgatcccctggagatgaagttacgtgaagccacctgatcacctgatgtggcaggctgcagggaaataagctctagttctctggaagaacaaatatttaactgggagagtggtttttattcctgtgaaataatagaacttgtggagagagcccctgaggtcacacacctagtgcttctgggaaagaggagggaccacagtctcttgtttttgtgtcacactggagattaatctgggacttcctggctccagatggaggctatcttccctgagcataaccccctaacctacgtgggggtgggggaagatgtcaagtgttatcacaggagacttgagagg

SEQ ID NO: 3 - DNA, 928 bp, Artificial Sequence
  Synthetic polynucleotide
aagtacctcagcctccagcagatgcaagaactctcctccattctgaacctgagctataagcaggtggggtccttagaagttgctgtaatgtaacacaatgagactgacttgctaatgaatcgaactaacgtctggacgtctctctaactatcctatcctcctatcctatgcgcctgttaaaatcataattatgtgattgattgattgattgattatgtataagtacactatagctgtcttcagatacaccagaagagggcatcagatctcattacggatggttgtgagccaccatgtggttgctgggatttgaactcaggaccttcggaagagcagtcagtgctcttaaccgctgagccatctctccagccccaattatgtgatttatatatttcccagtttgtttcattggttactttatcttctacttcacctcccacacccatccccaaatcagggtctcactgtagccctgactggtctctgtagaccaggctgggagaggttcacctgcctctgtctcctgggtgctggaaataaaggtgtgcctggcttttttcctttcttttcttttttaagattgtttatttttaaattcctttagttcattggtgttttgcctgcatgtgtttgtctttgtgagagtatcagatctcctggactagagttacagacagtcgtgagctgccatgtggccgctgagaattgaacccaggtcctctggaagagcagccagtgctcttaacagaagagccatttcccccagcacctgtctttttcaagaaagagtttcactttacaggcctggctgtcatgtttaaggtcgggctgtagacctgtctgcattctgtatgtcttagcttgttcgaatgtcctgtcactctgcaggttaagacctggtttcaaaaccaaaggatgaagtgcaagc

SEQ ID NO: 4 - DNA, 5955 bp, Mus musculus
ttaccaacctggacaacacaagatggaatactgtgctctgaaaacgcagagccagcacttctctggggtctctggggacatatctggttggggctcggggtcccatggtgtagagcctctaaactctggaggactggaggtgcaatggctgtcttgtcctggccttggacatgggctgaaatactgggttcacccatatctaggactctagacgggtgggtaagcaagaactgaggagtggccccagaaataattggcacacgaacattcaatggatgttttaggctctccagaggatggctgagtgggctgtaaggacaggccgagagggtgcagtgccaacaggctttgtggtgcgatggggcatccgagcaactggtttgtgaggtgtccggtgacccaaggcaggggtgagaggaccttgaaggttgaaaatgaaggcctcctggggtcccgtcctaagggttgtcctgtccagacgtccccaacctccgtctggaagacacaggcagatagcgctcgcctcagtttctcccacccccacagctctgctcctccacccacccagggggcggggccagaggtcaaggctagagggtgggattggggagggagaggtgaaaccgtccctaggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggaggtaagtgaagggacttggctgggctggcagaggcagcagtgaagggaattgggaacatgtagggtagccaccctgcctgccaaaggtggtgatggctgccgggcctcctgagaagcacgacgcagtgtggactagaacccagaattgcaagaatcagaaaccggcctggattgtttcggcctggcccttgtcatgtaggtcacctaggcctggcctgtgtcccgacacttgcttcatgccatcactgtctgtacaccagtgatgcgtgaaaatcagcccccccccaaaaaaaaaaacatatcagcccctctggggacttggatcacagtcggacccaggaacttggccttaaggttaggcatggctgggggggtaaaaaatggtgcttatcctggagttattgttactgaagaggttgggtgtgactggctgctgataggagctcttgtttgggccatgtgtggagtagggctcaccttcagtcaagtttacggcctgtctactttagcctcagactccatgagtcacctttacacgagcagacccttgtagtgcctgaggtgcagatctgatcgatttcagcctttctacctttccttgtaaacaagaaagggacacccttgggtaggggagttttatctccaggccatcttaagatcattctgtgagtgcacgggccttgcttagtgtctgatggcctacagccagcactctggagcaagtgtaagcaattagccttaagaacaaggtgcgagtggataccgatgcccgccgggagttccgacagcttagcgattgttgtagcaggagtcccctccctaagtgccagtttctgtgttatctcaggtcctgtatgccgccgggagtcccctaggaaggcattaatagtttatctcacatcttaaatggcccttaatgaagcaagagatttgaaccttagttaagctaatcccaaatcctcaaaataggatttagaaaagccaaagacactgctgagggcgattacaagttttggtcttttgaggagcagttggagatgaaagtctgtctgaagccgagagaatccttttccattgaaatggcattgaggtgtgcctcactggctgctgcttctgtctgtgccctgggttggccagcctttgtggagcacctcagccctccatcctggacctttgctccaacaacctgctcctcttccgccctcaaggctgacttgcatctccccagatgactgcctccatttctgtcttctgttagagacagaaaagcctgagaaaccgacagccattttggggggggggggtccggttcacacgctgcaacttagaaagcacactcaactggccatctgttataccctccccacctggtcccaaccatcactgtgtactactgagaagaaggcagccttagccacaccctcgagtgcccctgccgttctattgctcatacatcgattgatatccctgtttcaactttgaaaaaaaaaaatttttttttttttgtggtgtgtgcatgcctgctactgtacacctgtgggcgtcagaggtggtcctctgcaccctccggccagtaccgcatccagggtgagtcagatgatttcctgtggtttgggcctcaaggcttctcacctccagaggcttctagcctgctgccttgctttctctgtcgcactctagtacagcaggagttttcttcgcactccggagtgttgtcagctcctggggcatggacatttggctacttagagtgtgctgtgtaggttttcatttagagctgaacagagggatggatcttattaccccagcccttgagacactgaggcaggagagcttcctagtgagtccctgtttcaatatcttcactaatactgtgtcatactttgggactttctttcttcctttctttcttttgattttttttttttttatatgagtacagtgtacctgtcttcagacacacaccagaagagggcatcagatcctactacagaaggttgtgagccaccttgtggttgctgggaattgaactccggatctccggaagagaagtccgtataccaacttctgtattagtcagggttctctagagtcacagaacttatggacagtctctagatagtaaaggaatttattgatgacttacagtcggcagcccaattcccaacaatggttcagtcgcagctgtgaatggaagtccaaggatctagcagttacttagtctcacgcagcaagcaggcgaaggagcaagagctagagcttaactgctgagccatgtgtttcttgagtaaagggattacatgctcgttcgtctggtcaattctgcagccttaaaacttcttcagaatagggtgacattttgtcctcagtggggcggttttgagtaatctgtgagcagataggaacttgctggggtactgcacagaactctgggtagtgtggtactgtagatggctaggttctggggggggaaagagccatctatgtcacctaggaatagagtgaataacatttatataatcagaccagcccttgaggaggctgagatcttttcatggggcaccctagggtcacagtcccagctggtgtgactctgacaagtctgcctttctcactacagtcccaggacatgaaagccctgcagaaggagctagaacagtttgccaagctgctgaagcagaagaggatcaccttggggtacacccaggccgacgtggggctcaccctgggcgttctctttggtgggtctcccccagcatgttctgatctcacggctcttaatgtaggcgcaagggggtggggcatcttaggagctgcttctccacaggtaagggaggattagacgcttgtagcttgaactgtcagaggtgggggcttgggctcccttcttgctgcctcactcactctgtttgatcggcctttcaggaaaggtgttcagccagaccaccatctgtcgcttcgaggccttgcagctcagccttaagaacatgtgtaagctgcggcccctgctggagaagtgggtggaggaagccgacaacaatgagaaccttcaggaggtgaggagtggcaggatgtgtgcaatgtctgccaggcacagtcccttctgctgcttccattcctggcttgaaactcctccctctccaaccggagctcgcaggagaagttctgtgtccttattctgctgctatgaattggaatccagagccttaacatttgctaatcaatcaggctctctccttctgagtcaccctctgcccccaccagcctgacaatggtccctccccagaaccccgtctagtgctggtgaaggctcagacctaggtctaccagccccttccagagcccctttcagtaacccctggctctggggccacatccagtcaatgctcccttagcacaatcccttagcggtttgttcttcagtcccatctcaaggtggggctgttgccaagtcaaatactaaagttgctcttgtcgcccccatcttcccctgcccagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtgagtgccaagatcctgccctgtggtacctggatgtttccctgttcccattccccaccccccccacccccccacccccaccgccgccaccgctgactgcagcatcccagagcttatgatctgatgtccatctctgtgcccatcctaggtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagctgaattttgttttccttcagttcctagagagcctgcggttggagaaagccagtaatggattctcaaaccccaggtgatcttcaaaacaggcgccattgaaaccattggagttccacaaaatgcccagggatagttggggttggagcccaacctatagaggaaggcattgcatattcgccatcctagaggcggtaagtctctgctagctgatggacatcacctcatagccattgtctggcagccgccttctttcctcttgtcactctgggagttctggtgggcttatactttaaaaaaaagagtttttttgggggggttaagatttattttatttatatgggtacactgtagctgtcttctagacacaccagaagagggcatgggatcccattacagatggttgtgagccaccatgtggttgctgggaattgaactcaggacctctggaagagcagtcagtgctcttaaccgctgagccatctctccagccctcaaactcttttttttcttttccttcaagatgagttctgtgtagtcctggcggaccaggttggcctcagatcagcctgcctctgcctccgcagtgctgagattaa

SEQ ID NO: 5 - DNA, 20 bp, Artificial Sequence
  Synthetic polynucleotide
ctacagtcccaggacatgaa

SEQ ID NO: 6 - DNA, 20 bp, Artificial Sequence
  Synthetic polynucleotide
ctccacctcagacctctggt

SEQ ID NO: 7 - DNA, 20 bp, Artificial Sequence
  Synthetic polynucleotide
atgatggagacggagctgaa

SEQ ID NO: 8 - DNA, 20 bp, Artificial Sequence
  Synthetic polynucleotide
gtgttgagcctctagtcgtt

SEQ ID NO: 9 - DNA, 20 bp, Artificial Sequence
  Synthetic polynucleotide
aagtacctcagcctccagca

SEQ ID NO: 10 - DNA, 20 bp, Artificial Sequence
  Synthetic polynucleotide
ggtttcctacttcacgttcg

SEQ ID NO: 11 - DNA, 20 bp, Artificial Sequence
  Synthetic polynucleotide
cgacttctacgaggtggagc

SEQ ID NO: 12 - DNA, 21 bp, Artificial Sequence
  Synthetic polynucleotide
ctgtagtcgccggatgtagct

SEQ ID NO: 13 - DNA, 20 bp, Artificial Sequence
  Synthetic polynucleotide
caccatgcccctcaacgtga

SEQ ID NO: 14 - DNA, 18 bp, Artificial Sequence
  Synthetic polynucleotide
gctttgagaccacgtatt

SEQ ID NO: 15 - DNA, 20 bp, Artificial Sequence
  Synthetic polynucleotide
cgtatccccgtagatggcag

SEQ ID NO: 16 - DNA, 20 bp, Artificial Sequence
  Synthetic polynucleotide
atgatcgactggtgggaccg

SEQ ID NO: 17 - DNA, 22 bp, Artificial Sequence
  Synthetic polynucleotide
agagcataccaacgtccaatgc

SEQ ID NO: 18 - DNA, 22 bp, Artificial Sequence
  Synthetic polynucleotide
gtgatgaccctaaagaggcgtg

SEQ ID NO: 19 - DNA, 22 bp, Artificial Sequence
  Synthetic polynucleotide
cgtggaagacgaggttacgctg

SEQ ID NO: 20 - DNA, 23 bp, Artificial Sequence
  Synthetic polynucleotide
cggatactgtgtgtggtaagtcg

SEQ ID NO: 21 - DNA, 31 bp, Artificial Sequence
  Synthetic polynucleotide
atcccagaacgggtccagcggtggcaaccac

SEQ ID NO: 22 - DNA, 30 bp, Artificial Sequence
  Synthetic polynucleotide
caatagtgaggagttgtttacgtcgtctag

SEQ ID NO: 23 - DNA, 20 bp, Artificial Sequence
  Synthetic polynucleotide
ggtgcagcttgcagcagtaa

SEQ ID NO: 24 - DNA, 24 bp, Artificial Sequence
  Synthetic polynucleotide
ttgctggaggacctggatctgaaa

SEQ ID NO: 25 - DNA, 22 bp, Artificial Sequence
  Synthetic polynucleotide
gtgacgaggcccagagcaagag

SEQ ID NO: 26 - DNA, 22 bp, Artificial Sequence
  Synthetic polynucleotide
ctcatgctactcaggccgggga

SEQ ID NO: 27 - DNA, 21 bp, Artificial Sequence
  Synthetic polynucleotide
catcaccatcttccaggagcg

SEQ ID NO: 28 - DNA, 20 bp, Artificial Sequence
  Synthetic polynucleotide
ggatgggggttacacaggca